(19) United States
(12) Patent Application Publication
(10) Pub. No.: US 2018/0256623 A1
(43) Pub. Date: Sep. 13, 2018

(54) COMPOSITIONS AND METHOD FOR REGULATING ADIPOSE TISSUE LIPOLYSIS, INSULIN-RESISTANCE, AND HYPERGLYCEMIA

(71) Applicant: University of Virginia Patent Foundation, Charlottesville, VA (US)

(72) Inventors: Thurl E. Harris, JR., Covesville, VA (US); Vidisha B. Raje, Charlottesville, VA (US); Alexandra KadI, Charlottesville, VA (US); Garrett R. Mullins, Charlottesville, VA (US)

(21) Appl. No.: 15/760,990

(22) PCT Filed: Sep. 16, 2016
(86) PCT No.: PCT/US2016/052298

§ 371 Date: Mar. 16, 2018

Related U.S. Application Data
(60) Provisional application No. 62/219,295, filed on Sep. 16, 2015.

(51) Int. Cl. A61K 31/7076; A61P 3/06; A61P 3/10; A61P 17/02; A61K 31/17

(52) U.S. Cl. CPC A61K 31/7076; A61P 3/06; A61K 9/0019; A61P 17/02; A61K 31/17; A61P 3/10

(57) ABSTRACT

The present application discloses compositions and methods useful for controlling blood glucose and insulin resistance during the acute stress response. It is shown that Atglistatin can attenuate glucose excursions and insulin resistance arising from surgical procedures. It is further disclosed herein that Atglistatin is useful for inhibiting and preventing hyperglycemia and insulin resistance in many acute stress conditions, including, but not limited to, surgery, trauma/hemorrhage, burns, sepsis, myocardial infarction, and stroke.

### CROSS-REFERENCE TO RELATED APPLICATIONS

This application claims the priority of U.S. Provisional Application Ser. No. 62/219,295, filed Sep. 16, 2015, the disclosure of which is incorporated by reference herein in its entirety.

### STATEMENT OF GOVERNMENT SUPPORT

This invention was made with government support under Grant No. DK 101946, awarded by The National Institutes of Health. The government has certain rights in the invention.

### BACKGROUND

Postoperative and trauma-induced hyperglycemia is a common occurrence, even in patients without a previous history of diabetes, and is associated with worse neurologic outcome, increased risk of nosocomial infections, prolonged ICU and hospital length, and increased overall mortality. Blood glucose levels typically peak during and immediately after surgery, but can remain highly elevated for days to weeks depending on the severity of the injury and/or contributing illness.

Diabetic patients that undergo elective or emergency surgery have higher postoperative glycemic excursions and exhibit elevated rates of morbidity and mortality compared to non-diabetic controls.

Insulin therapy is generally effective, but requires close monitoring and can cause episodes of hypoglycemia-particularly under postoperative conditions and in diabetic patients. Although options other than insulin therapy are beginning to be explored, none are likely to be effective in patients with T1DM (Type 1 Diabetes Mellitus).

Attention to pre-operative glucose levels in diabetics can help prevent extensive postoperative hyperglycemia, but strict guidelines frequently disqualify many patients with poorly controlled diabetes for elective surgery and cannot be applied to emergency surgery situations.

Trauma, hemorrhage and shock can cause lung injury and increase susceptibility to pneumonia and delay wound healing. Patients that develop early post-traumatic pneumonia have increased duration of mechanical ventilation, delayed recovery, and increased mortality and require substantially higher health care costs.

### SUMMARY

Diabetic patients, that undergo surgery, including elective or emergency surgery, have higher postoperative morbidity and mortality rates than nondiabetic controls. Hyperglycemia is a common occurrence following trauma or surgery, even in patients without a previous history of diabetes. It is well accepted that postoperative and post trauma hyperglycemia is associated with worse neurologic outcome, increased risk of nosocomial infections, prolonged ICU and hospital length, and increased overall mortality. Current treatment involves intensive insulin therapy; however, close and accurate monitoring of blood glucose levels is required for both healthy and especially diabetic patients due to the risk of hypoglycemia. Patients with type I diabetes mellitus are also at risk from hyperglycemic induction of diabetic ketoacidosis. The seriousness of postoperative hyperglycemia, the risks of insulin therapy initiated hypoglycemia, and the lack of alternatives to insulin for T1DM patients warrants the need for understanding the molecular mechanisms involved to allow the development of alternatives to insulin for the prevention and treatment of hyperglycemia.

There is a long felt need in the art for compositions and methods to regulate adipose tissue lipolysis, insulin-resistance, and hyperglycemia, particularly in a perioperative setting. The present invention satisfies these needs.

It is disclosed herein that inhibition of ATGL (adipose triacylglycerol lipase), resulting in reducing adipose tissue lipolysis, is useful for treating or preventing varying diseases, disorders, and conditions as well as stress, including stress associated with surgery. In one aspect, the compositions and methods are useful for treating and preventing hyperglycemia associated with surgery. In one aspect, the compositions and methods of the invention are useful for preventing and treating trauma-induced hyperglycemia.

One embodiment provides a method to prevent or treat stress related adipose tissue lipolysis, insulin-resistance, and/or hyperglycemia comprising administering an effective amount of a compound of formula (II) to a subject in need thereof

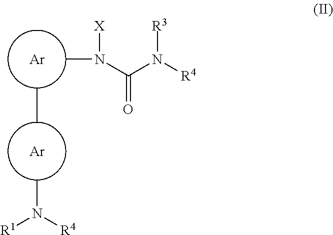

wherein R¹, R², R³, R⁴ and X are individually H, (C₁-C₆) alkyl, (C₃-C₆)cycloalkyl, (C₁-C₆)alkanoyl, trifluoromethyl, hydroxy (C₁-C₆)alkyl, aryl, aryl(C₁-C₆)alkyl;
each Ar is individually selected from aryl; or a pharmaceutically acceptable salt thereof.

Another embodiment provides a method to prevent or treat stress related adipose tissue lipolysis, insulin-resistance, and/or hyperglycemia comprising administering an effective amount of a compound of formula (III) to a subject in need thereof

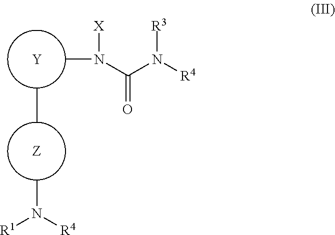

wherein R¹, R², R³, R⁴ and X are individually H, (C₁-C₆) alkyl, (C₃-C₆)cycloalkyl, (C₁-C₆)alkanoyl, trifluoromethyl, hydroxy (C₁-C₆)alkyl, aryl, aryl(C₁-C₆)alkyl;
Y and Z are selected from aryl or Het, such as heteroaryl and wherein Y and Z comprise a bond connecting two carbon atoms of Y and Z; or a pharmaceutically acceptable salt thereof.

Another embodiment provides a method to prevent or treat stress related adipose tissue lipolysis, insulin-resistance, and/or hyperglycemia comprising administering an effective amount of a compound of formula (I) to a subject in need thereof

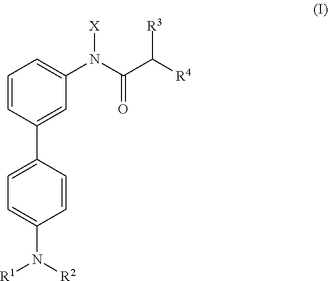

wherein R¹, R², R³, R⁴ and X are individually selected from H, (C₁-C₆) alkyl, phenyl, benzyl, phenylethyl or a pharmaceutically acceptable slat thereof.

One embodiment provides a method to inhibit or treat postoperative hyperglycemia comprising administering an effective amount of a compound of formula (I), (II), or (III) to a subject in need thereof.

Another embodiment provides a method to improve wound healing, decrease postoperative morbidities, shorten hospital stay and/or improve mortality due to trauma and/or surgery comprising administering an effective amount of a compound of formula (I), (II), or (III) to a subject in need thereof.

In one embodiment, the compound is

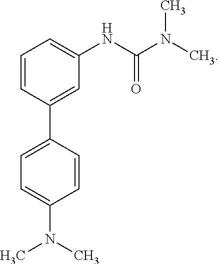 [SMILES: CN(C)C(=O)Nc1cccc(-c2ccc(N(C)C)cc2)c1]

In one embodiment, the subject is diabetic. In another embodiment, the subject is not diabetic.

In one embodiment, the stress is induced by trauma. In one embodiment, the trauma is a result of hemorrhage, burn, sepsis, myocardial infarction, and/or stroke. In one embodiment, the stress is induced by surgery. In another embodiment, the stress is induced by anesthesia. In one embodiment, the compound is administered before, during and/or after the stress, the trauma and/or surgery.

In one embodiment, insulin is not administered; another embodiment further comprises the administering of insulin to the subject.

In one embodiment, the present invention provides compositions and methods for inhibiting hyperglycemia. In one aspect, it is useful for treating postoperative hyperglycemia (hyperglycemia that arises after surgery). The compositions and methods of the invention provide an alternative to the use of insulin for prevention and treatment of hyperglycemia.

In one embodiment, the present invention provides compositions and methods useful for inhibiting adipose tissue lipolysis. In one aspect, it is stress-induced adipose tissue lipolysis. In one aspect, the stress is an acute stress response.

In one embodiment, the compositions and methods of the invention are useful for controlling blood glucose during a stress response.

In one aspect, a stress response is a response to surgery.

In one embodiment, inhibiting stress-induced adipose tissue lipolysis with the compounds and methods of the invention is useful for treating and preventing insulin resistance and hyperglycemia. In one aspect, the insulin resistance is whole body insulin resistance. In one aspect, the method reduces blood glucose.

Based on the teachings herein, one of ordinary skill in the art will appreciate that various types of molecules can be used in the practice of the invention, as long as they have the same activity (or substantially similar) as that desired for regulating the processes described herein.

In one embodiment of the invention, the compositions and methods are useful for inhibiting adipose lipolysis to attenuate postoperative hyperglycemia and for limiting postoperative hyperglycemia to improve wound healing, decrease postoperative morbidities, such as infection, shorten hospital stay and to improve mortality.

In one embodiment, the compositions and methods of the invention are useful improving healing. In another embodiment, they are useful for decreasing infection.

The compositions and methods of the invention can be used in a perioperative setting or following trauma.

In one aspect, a subject is a normal subject (e.g., a non-diabetic subject). In another aspect, a subject is diabetic (Type 1 or 2).

Various aspects and embodiments of the invention are described in further detail below.

### BRIEF DESCRIPTION OF THE DRAWINGS

FIG. 1 depicts the hemorrhage protocol on streptozotocin (STZ)-treated mice to mimic surgical induction of the neuroendocrine stress response in humans.

FIGS. 2A-B depict demonstrate the effect of Atglistatin pre-treatment on hyperglycemia in control mice is shown in FIG. 2A. FIG. 2B demonstrates the effectiveness of the Atglistatin in attenuating adipose tissue lipolysis as assessed by serum glycerol levels.

FIGS. 3A-B provide a summary of the hyperglycemia attenuation by Atglistatin is shown FIG. 3A, and the attenuation of insulin resistance in adipose tissue and skeletal muscle in FIG. 3B.

FIGS. 4A-B provide a Pseudomonas infection model which demonstrates induction of glycerol release and hyperglycemia.

### DETAILED DESCRIPTION

### Abbreviations and Acronyms

ATGL—adipose triacylglycerol lipase (NM_020376; NP_065109.1)

DCC—Diabetic Complications Consortium, also referred to as DiaComp

DKA—diabetic ketoacidosis

HSL—hormone-sensitive lipase

MAP—mean arterial pressure

PNPLA2—patatin-like phospholipase domain-containing protein 2

STZ—streptozotocin

T1DM—Type 1 Diabetes Mellitus

TG—triglyceride

### Definitions

In describing and claiming the invention, the following terminology will be used in accordance with the definitions set forth below. Unless defined otherwise, all technical and scientific terms used herein have the commonly understood by one of ordinary skill in the art to which the invention pertains. Methods and materials similar or equivalent to those described herein may be useful in the practice or testing of the present invention.

The articles “a” and “an” are used herein to refer to one or to more than one (i.e., to at least one) of the grammatical object of the article. By way of example, “an element” means one element or more than one element.

The term “about,” as used herein, means approximately, in the region of, roughly, or around. When the term “about” is used in conjunction with a numerical range, it modifies that range by extending the boundaries above and below the numerical values set forth. In general, the term “about” is used herein to modify a numerical value above and below the stated value by a variance of 10%. In one aspect, the term “about” means plus or minus 20% of the numerical value of the number with which it is being used. Therefore, about 50% means in the range of 45%-55%. Numerical ranges recited herein by endpoints include all numbers and fractions subsumed within that range (e.g. 1 to 5 includes 1, 1.5, 2, 2.75, 3, 3.90, 4, and 5). It is also to be understood that all numbers and fractions thereof are presumed to be modified by the term “about.”

All percent compositions are given as weight-percentages, unless otherwise stated.

The terms “additional therapeutically active compound” or “additional therapeutic agent,” as used in the context of the present invention, refers to the use or administration of a compound for an additional therapeutic use for a particular injury, disease, or disorder being treated. Such a compound, for example, could include one being used to treat an unrelated disease or disorder, or a disease or disorder which may not be responsive to the primary treatment for the injury, disease or disorder being treated.

As use herein, the terms “administration of” and or “administering” a compound should be understood to mean providing a compound of the invention or a prodrug of a compound of the invention to a subject in need of treatment.

As used herein, the term “aerosol” refers to suspension in the air. In particular, aerosol refers to the particlization or atomization of a formulation of the invention and its suspension in the air.

As used herein, an “agent” is meant to include something being contacted with a cell population to elicit an effect, such as a drug, a protein, a peptide. An “additional therapeutic agent” refers to a drug or other compound used to treat an illness and can include, for example, an antibiotic or a chemotherapeutic agent.

As used herein, an “agonist” is a composition of matter which, when administered to a mammal such as a human, enhances or extends a biological activity attributable to the level or presence of a target compound or molecule of interest in the mammal.

The term “alkyne” or “alkyne group,” as used herein, refers to an unsaturated hydrocarbon containing at least one carbon-carbon triple bond between two carbon atoms.

As used herein, “alleviating a disease or disorder symptom,” means reducing the severity of the symptom or the frequency with which such a symptom is experienced by a patient, or both.

As used herein, amino acids are represented by the full name thereof, by the three letter code corresponding thereto, or by the one-letter code corresponding thereto, as indicated in the following table:

| Full Name | Three-Letter Code | One-Letter Code |
|---|---|---|
| Aspartic Acid | Asp | D |
| Glutamic Acid | Glu | E |
| Lysine | Lys | K |
| Arginine | Arg | R |
| Histidine | His | H |
| Tyrosine | Tyr | Y |
| Cysteine | Cys | C |
| Asparagine | Asn | N |
| Glutamine | Gln | Q |
| Serine | Ser | S |
| Threonine | Thr | T |
| Glycine | Gly | G |
| Alanine | Ala | A |
| Valine | Val | V |
| Leucine | Leu | L |
| Isoleucine | Ile | I |
| Methionine | Met | M |
| Proline | Pro | P |
| Phenylalanine | Phe | F |
| Tryptophan | Trp | W |

The expression “amino acid” as used herein is meant to include both natural and synthetic amino acids, and both D and L amino acids. “Standard amino acid” means any of the twenty standard L-amino acids commonly found in naturally occurring peptides. “Nonstandard amino acid residue” means any amino acid, other than the standard amino acids, regardless of whether it is prepared synthetically or derived from a natural source. “Nonstandard amino acids” include “non-natural” and “noncanonical” amino acids. As used herein, “synthetic amino acid” also encompasses chemically modified amino acids, including but not limited to salts, amino acid derivatives (such as amides), and substitutions. Amino acids contained within the peptides of the present invention, and particularly at the carboxy- or amino-terminus, can be modified by methylation, amidation, acetylation or substitution with other chemical groups which can change the peptide's circulating half-life without adversely affecting their activity. Additionally, a disulfide linkage may be present or absent in the peptides of the invention.

The term “amino acid” is used interchangeably with “amino acid residue,” and may refer to a free amino acid and to an amino acid residue of a peptide. It will be apparent from the context in which the term is used whether it refers to a free amino acid or a residue of a peptide.

Amino acids have the following general structure:

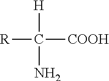

Amino acids may be classified into seven groups on the basis of the side chain R: (1) aliphatic side chains, (2) side chains containing a hydroxylic (OH) group, (3) side chains containing sulfur atoms, (4) side chains containing an acidic or amide group, (5) side chains containing a basic group, (6) side chains containing an aromatic ring, and (7) proline, an imino acid in which the side chain is fused to the amino group.

The nomenclature used to describe the peptide compounds of the present invention follows the conventional practice wherein the amino group is presented to the left and the carboxy group to the right of each amino acid residue. In the formulae representing selected specific embodiments of the present invention, the amino- and carboxy-terminal groups, although not specifically shown, will be understood to be in the form they would assume at physiologic pH values, unless otherwise specified.

The term “basic” or “positively charged” amino acid as used herein, refers to amino acids in which the R groups have a net positive charge at pH 7.0, and include, but are not limited to, the standard amino acids lysine, arginine, and histidine.

As used herein, an “analog”, or “analogue” of a chemical compound is a compound that, by way of example, resembles another in structure but is not necessarily an isomer (e.g., 5-fluorouracil is an analog of thymine).

The term “antibody,” as used herein, refers to an immunoglobulin molecule which is able to specifically bind to a specific epitope on an antigen. Antibodies can be intact immunoglobulins derived from natural sources or from recombinant sources and can be immunoreactive portions of intact immunoglobulins. Antibodies are typically tetramers of immunoglobulin molecules. The antibodies in the present invention may exist in a variety of forms including, for example, polyclonal antibodies, monoclonal antibodies, Fv, Fab and F(ab)₂, as well as single chain antibodies and humanized antibodies.

An “antibody heavy chain,” as used herein, refers to the larger of the two types of polypeptide chains present in all antibody molecules.

An “antibody light chain,” as used herein, refers to the smaller of the two types of polypeptide chains present in all antibody molecules.

By the term “synthetic antibody” as used herein, is meant an antibody which is generated using recombinant DNA technology, such as, for example, an antibody expressed by a bacteriophage as described herein. The term should also be construed to mean an antibody which has been generated by the synthesis of a DNA molecule encoding the antibody and which DNA molecule expresses an antibody protein, or an amino acid sequence specifying the antibody, wherein the DNA or amino acid sequence has been obtained using synthetic DNA or amino acid sequence technology which is available and well known in the art.

An “antagonist” is a composition of matter which when administered to a mammal such as a human, inhibits a biological activity attributable to the level or presence of a compound or molecule of interest in the mammal.

The term “antigen” as used herein is defined as a molecule that provokes an immune response. This immune response may involve either antibody production, or the activation of specific immunologically-competent cells, or both. An antigen can be derived from organisms, subunits of proteins/antigens, killed or inactivated whole cells or lysates.

The term “antigenic determinant” as used herein refers to that portion of an antigen that makes contact with a particular antibody (i.e., an epitope). When a protein or fragment of a protein, or chemical moiety is used to immunize a host animal, numerous regions of the antigen may induce the production of antibodies that bind specifically to a given region or three-dimensional structure on the protein; these regions or structures are referred to as antigenic determinants. An antigenic determinant may compete with the intact antigen (i.e., the “immunogen” used to elicit the immune response) for binding to an antibody.

The term “antimicrobial agents” as used herein refers to any naturally-occurring, synthetic, or semi-synthetic compound or composition or mixture thereof, which is safe for human or animal use as practiced in the methods of this invention, and is effective in killing or substantially inhibiting the growth of microbes. “Antimicrobial” as used herein, includes antibacterial, antifungal, and antiviral agents.

As used herein, the term “antisense oligonucleotide” or antisense nucleic acid means a nucleic acid polymer, at least a portion of which is complementary to a nucleic acid which is present in a normal cell or in an affected cell. “Antisense” refers particularly to the nucleic acid sequence of the non-coding strand of a double stranded DNA molecule encoding a protein, or to a sequence which is substantially homologous to the non-coding strand. As defined herein, an antisense sequence is complementary to the sequence of a double stranded DNA molecule encoding a protein. It is not necessary that the antisense sequence be complementary solely to the coding portion of the coding strand of the DNA molecule. The antisense sequence may be complementary to regulatory sequences specified on the coding strand of a DNA molecule encoding a protein, which regulatory sequences control expression of the coding sequences. The antisense oligonucleotides of the invention include, but are not limited to, phosphorothioate oligonucleotides and other modifications of oligonucleotides.

An “aptamer” is a compound that is selected in vitro to bind preferentially to another compound (for example, the identified proteins herein). Often, aptamers are nucleic acids or peptides because random sequences can be readily generated from nucleotides or amino acids (both naturally occurring and/or synthetically made) in large numbers but of course they need not be limited to these.

The term “binding” refers to the adherence of molecules to one another, such as, but not limited to, enzymes to substrates, ligands to receptors, antibodies to antigens, DNA binding domains of proteins to DNA, and DNA or RNA strands to complementary strands.

“Binding partner,” as used herein, refers to a molecule capable of binding to another molecule.

The term “biocompatible,” as used herein, refers to a material that does not elicit a substantial detrimental response in the host.

fAs used herein, the term “biologically active fragments” or “bioactive fragment” of the polypeptides encompasses natural or synthetic portions of the full-length protein that are capable of specific binding to their natural ligand or of performing the function of the protein.

The term “biological sample,” as used herein, refers to samples obtained from a subject, including, but not limited to, skin, hair, tissue, blood, plasma, cells, sweat and urine.

As used herein, the term “chemically conjugated,” or “conjugating chemically” refers to linking the antigen to the carrier molecule. This linking can occur on the genetic level using recombinant technology, wherein a hybrid protein may be produced containing the amino acid sequences, or portions thereof, of both the antigen and the carrier molecule. This hybrid protein is produced by an oligonucleotide sequence encoding both the antigen and the carrier molecule, or portions thereof. This linking also includes covalent bonds created between the antigen and the carrier protein using other chemical reactions, such as, but not limited to glutaraldehyde reactions. Covalent bonds may also be created using a third molecule bridging the antigen to the carrier molecule. These cross-linkers are able to react with groups, such as but not limited to, primary amines, sulfhydryls, carbonyls, carbohydrates, or carboxylic acids, on the antigen and the carrier molecule. Chemical conjugation also includes non-covalent linkage between the antigen and the carrier molecule.

The term “competitive sequence” refers to a peptide or a modification, fragment, derivative, or homolog thereof that competes with another peptide for its cognate binding site.

“Complementary” as used herein refers to the broad concept of subunit sequence complementarity between two nucleic acids, e.g., two DNA molecules. When a nucleotide position in both of the molecules is occupied by nucleotides normally capable of base pairing with each other, then the nucleic acids are considered to be complementary to each other at this position. Thus, two nucleic acids are complementary to each other when a substantial number (at least 50%) of corresponding positions in each of the molecules are occupied by nucleotides which normally base pair with each other (e.g., A:T and G:C nucleotide pairs). Thus, it is known that an adenine residue of a first nucleic acid region is capable of forming specific hydrogen bonds (“base pairing”) with a residue of a second nucleic acid region which is antiparallel to the first region if the residue is thymine or uracil. Similarly, it is known that a cytosine residue of a first nucleic acid strand is capable of base pairing with a residue of a second nucleic acid strand which is antiparallel to the first strand if the residue is guanine. A first region of a nucleic acid is complementary to a second region of the same or a different nucleic acid if, when the two regions are arranged in an antiparallel fashion, at least one nucleotide residue of the first region is capable of base pairing with a residue of the second region. Preferably, the first region comprises a first portion and the second region comprises a second portion, whereby, when the first and second portions are arranged in an antiparallel fashion, at least about 50%, and preferably at least about 75%, at least about 90%, or at least about 95% of the nucleotide residues of the first portion are capable of base pairing with nucleotide residues in the second portion. More preferably, all nucleotide residues of the first portion are capable of base pairing with nucleotide residues in the second portion.

A “compound,” as used herein, refers to any type of substance or agent that is commonly considered a drug, or a candidate for use as a drug, as well as combinations and mixtures of the above.

As used herein, “conjugation” refers to the process of linking a molecule or substance, such as a fatty acid chain or therapeutic protein, to another molecule, such as a protein or carrier molecule, via p-orbital overlap. This linking can occur on the genetic level using recombinant technology, wherein a hybrid protein may be produced containing the amino acid sequences, or portions thereof, of both an antigen and carrier molecule. This hybrid protein is produced by an oligonucleotide sequence encoding both the antigen and the carrier molecule, or portions thereof. This linking can also occur via chemical reaction, including but not limited to, copper-catalyzed alkyne-azide cycloaddition.

As used herein, the term “conservative amino acid substitution” is defined herein as an amino acid exchange within one of the following five groups:

I. Small aliphatic, nonpolar or slightly polar residues:
Ala, Ser, Thr, Pro, Gly;

II. Polar, negatively charged residues and their amides:
Asp, Asn, Glu, Gln;

III. Polar, positively charged residues:
His, Arg, Lys;

IV. Large, aliphatic, nonpolar residues:
Met Leu, Ile, Val, Cys

V. Large, aromatic residues:
Phe, Tyr, Trp

A “control” cell is a cell having the same cell type as a test cell. The control cell may, for example, be examined at precisely or nearly the same time the test cell is examined. The control cell may also, for example, be examined at a time distant from the time at which the test cell is examined, and the results of the examination of the control cell may be recorded so that the recorded results may be compared with results obtained by examination of a test cell.

A “test” cell is a cell being examined.

The term “cycloaddition,” in the context of “copper-catalyzed alkyne-azide cycloaddition,” refers to an organic reaction catalyzed by copper in which an organic azide group reacts neatly with a terminal alkyne group to produce a triazole. The use of this chemical reaction includes, but is not limited to, the coupling of polymers with other polymers or small molecules.

As used herein, a “derivative” of a compound refers to a chemical compound that may be produced from another compound of similar structure in one or more steps, as in replacement of H by an alkyl, acyl, or amino group.

The use of the word “detect” and its grammatical variants refers to measurement of the species without quantification, whereas use of the word “determine” or “measure” with their grammatical variants are meant to refer to measurement of the species with quantification. The terms “detect” and “identify” are used interchangeably herein.

As used herein, a “detectable marker” or a “reporter molecule” is an atom or a molecule that permits the specific detection of a compound comprising the marker in the presence of similar compounds without a marker. Detectable markers or reporter molecules include, e.g., radioactive isotopes, antigenic determinants, enzymes, nucleic acids available for hybridization, chromophores, fluorophores, chemiluminescent molecules, electrochemically detectable molecules, and molecules that provide for altered fluorescence-polarization or altered light-scattering.

A “disease” is a state of health of an animal wherein the animal cannot maintain homeostasis, and wherein if the disease is not ameliorated then the animal's health continues to deteriorate.

In contrast, a “disorder” in an animal is a state of health in which the animal is able to maintain homeostasis, but in which the animal's state of health is less favorable than it would be in the absence of the disorder. Left untreated, a disorder does not necessarily cause a further decrease in the animal's state of health.

As used herein, the term “domain” refers to a part of a molecule or structure that shares common physicochemical features, such as, but not limited to, hydrophobic, polar, globular and helical domains or properties such as ligand binding, signal transduction, cell penetration and the like. Specific examples of binding domains include, but are not limited to, DNA binding domains and ATP binding domains.

As used herein, an “effective amount” or “therapeutically effective amount” means an amount sufficient to produce a selected effect, such as alleviating symptoms of a disease or disorder. In the context of administering compounds in the form of a combination, such as multiple compounds, the amount of each compound, when administered in combination with another compound(s), may be different from when that compound is administered alone. Thus, an effective amount of a combination of compounds refers collectively to the combination as a whole, although the actual amounts of each compound may vary. The term “more effective” means that the selected effect is alleviated to a greater extent by one treatment relative to the second treatment to which it is being compared.

The term “elixir,” as used herein, refers in general to a clear, sweetened, alcohol-containing, usually hydroalcoholic liquid containing flavoring substances and sometimes active medicinal agents.

As used in the specification and the appended claims, the terms “for example,” “for instance,” “such as,” “including” and the like are meant to introduce examples that further clarify more general subject matter. Unless otherwise specified, these examples are provided only as an aid for understanding the invention, and are not meant to be limiting in any fashion.

“Encoding” refers to the inherent property of specific sequences of nucleotides in a polynucleotide, such as a gene, a cDNA, or an mRNA, to serve as templates for synthesis of other polymers and macromolecules in biological processes having either a defined sequence of nucleotides (i.e., rRNA, tRNA and mRNA) or a defined sequence of amino acids and the biological properties resulting therefrom. Thus, a gene encodes a protein if transcription and translation of mRNA corresponding to that gene produces the protein in a cell or other biological system. Both the coding strand, the nucleotide sequence of which is identical to the mRNA sequence and is usually provided in sequence listings, and the non-coding strand, used as the template for transcription of a gene or cDNA, can be referred to as encoding the protein or other product of that gene or cDNA.

An “enhancer” is a DNA regulatory element that can increase the efficiency of transcription, regardless of the distance or orientation of the enhancer relative to the start site of transcription.

The term “epitope” as used herein is defined as small chemical groups on the antigen molecule that can elicit and react with an antibody. An antigen can have one or more epitopes. Most antigens have many epitopes; i.e., they are multivalent. In general, an epitope is roughly five amino acids or sugars in size. One skilled in the art understands that generally the overall three-dimensional structure, rather than the specific linear sequence of the molecule, is the main criterion of antigenic specificity.

As used herein, an “essentially pure” preparation of a particular protein or peptide is a preparation wherein at least about 95%, and preferably at least about 99%, by weight, of the protein or peptide in the preparation is the particular protein or peptide.

The terms “formula” and “structure” are used interchangeably herein when referring to a specific structure of a compound. A generic formula represents a genus with optional moieties, not a specific structure.

A “fragment” or “segment” is a portion of an amino acid sequence, comprising at least one amino acid, or a portion of a nucleic acid sequence comprising at least one nucleotide. The terms “fragment” and “segment” are used interchangeably herein.

As used herein, the term “fragment,” as applied to a protein or peptide, can ordinarily be at least about 3-15 amino acids in length, at least about 15-25 amino acids, at least about 25-50 amino acids in length, at least about 50-75 amino acids in length, at least about 75-100 amino acids in length, and greater than 100 amino acids in length.

As used herein, the term “fragment” as applied to a nucleic acid, may ordinarily be at least about 20 nucleotides in length, typically, at least about 50 nucleotides, more typically, from about 50 to about 100 nucleotides, at least about 100 to about 200 nucleotides, even more preferably, at least about 200 nucleotides to about 300 nucleotides, yet even more preferably, at least about 300 to about 350, even more preferably, at least about 350 nucleotides to about 500 nucleotides, yet even more preferably, at least about 500 to about 600, even more preferably, at least about 600 nucleotides to about 620 nucleotides, yet even more preferably, at least about 620 to about 650, and most preferably, the nucleic acid fragment will be greater than about 650 nucleotides in length.

As used herein, a “functional” biological molecule is a biological molecule in a form in which it exhibits a property by which it is characterized. A functional enzyme, for example, is one that exhibits the characteristic catalytic activity by which the enzyme is characterized.

The term “general anesthetic” as used herein when referring to anesthetics, means those in use prior to the present invention and includes clinically used general anesthetics such as ketamine, nitrous oxide, isoflurane, propofol, and etomidate.

“Homologous” as used herein, refers to the subunit sequence similarity between two polymeric molecules, e.g., between two nucleic acid molecules, e.g., two DNA molecules or two RNA molecules, or between two polypeptide molecules. When a subunit position in both of the two molecules is occupied by the same monomeric subunit, e.g., if a position in each of two DNA molecules is occupied by adenine, then they are homologous at that position. The homology between two sequences is a direct function of the number of matching or homologous positions, e.g., if half (e.g., five positions in a polymer ten subunits in length) of the positions in two compound sequences are homologous then the two sequences are 50% homologous, if 90% of the positions, e.g., 9 of 10, are matched or homologous, the two sequences share 90% homology. By way of example, the DNA sequences 3′ATTGCC5′ and 3′TATGGC share 50% homology.

As used herein, “homology” is used synonymously with “identity.”

The determination of percent identity between two nucleotide or amino acid sequences can be accomplished using a mathematical algorithm. For example, a mathematical algorithm useful for comparing two sequences is the algorithm of Karlin and Altschul (1990, Proc. Natl. Acad. Sci. USA 87:2264-2268), modified as in Karlin and Altschul (1993, Proc. Natl. Acad. Sci. USA 90:5873-5877). This algorithm is incorporated into the NBLAST and XBLAST programs of Altschul, et al. (1990, J. Mol. Biol. 215:403-410), and can be accessed, for example at the National Center for Biotechnology Information (NCBI) world wide web site having the universal resource locator using the BLAST tool at the NCBI website. BLAST nucleotide searches can be performed with the NBLAST program (designated “blastn” at the NCBI web site), using the following parameters: gap penalty=5; gap extension penalty=2; mismatch penalty=3; match reward=1; expectation value 10.0; and word size=11 to obtain nucleotide sequences homologous to a nucleic acid described herein. BLAST protein searches can be performed with the XBLAST program (designated “blastn” at the NCBI web site) or the NCBI “blastp” program, using the following parameters: expectation value 10.0, BLOSUM62 scoring matrix to obtain amino acid sequences homologous to a protein molecule described herein. To obtain gapped alignments for comparison purposes, Gapped BLAST can be utilized as described in Altschul et al. (1997, Nucleic Acids Res. 25:3389-3402). Alternatively, PSI-Blast or PHI-Blast can be used to perform an iterated search which detects distant relationships between molecules (Id.) and relationships between molecules which share a common pattern. When utilizing BLAST, Gapped BLAST, PSI-Blast, and PHI-Blast programs, the default parameters of the respective programs (e.g., XBLAST and NBLAST) can be used.

The percent identity between two sequences can be determined using techniques similar to those described above, with or without allowing gaps. In calculating percent identity, typically exact matches are counted.

As used herein, the term “hybridization” is used in reference to the pairing of complementary nucleic acids. Hybridization and the strength of hybridization (i.e., the strength of the association between the nucleic acids) is impacted by such factors as the degree of complementarity between the nucleic acids, stringency of the conditions involved, the length of the formed hybrid, and the G:C ratio within the nucleic acids.

As used herein, the term “inhaler” refers both to devices for nasal and pulmonary administration of a drug, e.g., in solution, powder and the like. For example, the term “inhaler” is intended to encompass a propellant driven inhaler, such as is used to administer antihistamine for acute asthma attacks, and plastic spray bottles, such as are used to administer decongestants.

The term “inhibit,” as used herein, refers to the ability of a compound, agent, or method to reduce or impede a described function, level, activity, rate, etc., based on the context in which the term “inhibit” is used. Preferably, inhibition is by at least 10%, more preferably by at least 25%, even more preferably by at least 50%, and most preferably, the function is inhibited by at least 75%. The term “inhibit” is used interchangeably with “reduce” and “block.”

The term “inhibit a complex,” as used herein, refers to inhibiting the formation of a complex or interaction of two or more proteins, as well as inhibiting the function or activity of the complex. The term also encompasses disrupting a formed complex. However, the term does not imply that each and every one of these functions must be inhibited at the same time.

As used herein “injecting or applying” includes administration of a compound of the invention by any number of routes and means including, but not limited to, topical, oral, buccal, intravenous, intramuscular, intra-arterial, intramedullary, intrathecal, intraventricular, transdermal, subcutaneous, intraperitoneal, intranasal, enteral, topical, sublingual, vaginal, ophthalmic, pulmonary, or rectal means.

As used herein, an “instructional material” includes a publication, a recording, a diagram, or any other medium of expression which can be used to communicate the usefulness of the peptide of the invention in the kit for effecting alleviation of the various diseases or disorders recited herein. Optionally, or alternately, the instructional material may describe one or more methods of alleviating the diseases or disorders in a cell or a tissue of a mammal. The instructional material of the kit of the invention may, for example, be affixed to a container which contains the identified compound invention or be shipped together with a container which contains the identified compound. Alternatively, the instructional material may be shipped separately from the container with the intention that the instructional material and the compound be used cooperatively by the recipient.

An “isolated nucleic acid” refers to a nucleic acid segment or fragment which has been separated from sequences which flank it in a naturally occurring state, e.g., a DNA fragment which has been removed from the sequences which are normally adjacent to the fragment, e.g., the sequences adjacent to the fragment in a genome in which it naturally occurs. The term also applies to nucleic acids which have been substantially purified from other components which naturally accompany the nucleic acid, e.g., RNA or DNA or proteins, which naturally accompany it in the cell. The term therefore includes, for example, a recombinant DNA which is incorporated into a vector, into an autonomously replicating plasmid or virus, or into the genomic DNA of a prokaryote or eukaryote, or which exists as a separate molecule (e.g., as a cDNA or a genomic or cDNA fragment produced by PCR or restriction enzyme digestion) independent of other sequences. It also includes a recombinant DNA which is part of a hybrid gene encoding additional polypeptide sequence.

A “ligand” is a compound that specifically binds to a target receptor.

A “receptor” is a compound that specifically binds to a ligand.

A ligand or a receptor (e.g., an antibody) “specifically binds to” or “is specifically immunoreactive with” a compound when the ligand or receptor functions in a binding reaction which is determinative of the presence of the compound in a sample of heterogeneous compounds.

As used herein, the term “linkage” refers to a connection between two groups. The connection can be either covalent or non-covalent, including but not limited to ionic bonds, hydrogen bonding, and hydrophobic/hydrophilic interactions.

As used herein, the term “linker” refers to a molecule that joins two other molecules either covalently or noncovalently, e.g., through ionic or hydrogen bonds or van der Waals interactions, e.g., a nucleic acid molecule that hybridizes to one complementary sequence at the 5′ end and to another complementary sequence at the 3′ end, thus joining two non-complementary sequences.

The term “modulate,” as used herein, refers to changing the level of an activity, function, or process. The term “modulate” encompasses both inhibiting and stimulating an activity, function, or process.

The term “nucleic acid construct,” as used herein, encompasses DNA and RNA sequences encoding the particular gene or gene fragment desired, whether obtained by genomic or synthetic methods.

Unless otherwise specified, a “nucleotide sequence encoding an amino acid sequence” includes all nucleotide sequences that are degenerate versions of each other and that encode the same amino acid sequence. Nucleotide sequences that encode proteins and RNA may include introns.

The term “oligonucleotide” typically refers to short polynucleotides, generally, no greater than about 50 nucleotides. It will be understood that when a nucleotide sequence is represented by a DNA sequence (i.e., A, T, G, C), this also includes an RNA sequence (i.e., A, U, G, C) in which “U” replaces “T.”

By describing two polynucleotides as “operably linked” is meant that a single-stranded or double-stranded nucleic acid moiety comprises the two polynucleotides arranged within the nucleic acid moiety in such a manner that at least one of the two polynucleotides is able to exert a physiological effect by which it is characterized upon the other. By way of example, a promoter operably linked to the coding region of a gene is able to promote transcription of the coding region.

As used herein, “parenteral administration” of a pharmaceutical composition includes any route of administration characterized by physical breaching of a tissue of a subject and administration of the pharmaceutical composition through the breach in the tissue. Parenteral administration thus includes, but is not limited to, administration of a pharmaceutical composition by injection of the composition, by application of the composition through a surgical incision, by application of the composition through a tissue-penetrating non-surgical wound, and the like. In particular, parenteral administration is contemplated to include, but is not limited to, subcutaneous, intraperitoneal, intramuscular, intrasternal injection, and kidney dialytic infusion techniques.

The term “peptide” typically refers to short polypeptides.

The term “per application” as used herein refers to administration of a drug or compound to a subject.

As used herein, the term “perioperative” generally refers to the three phases of surgery: preoperative, intraoperative, and postoperative.

As used herein, the term “pharmaceutically acceptable carrier” includes any of the standard pharmaceutical carriers, such as a phosphate buffered saline solution, water, emulsions such as an oil/water or water/oil emulsion, and various types of wetting agents. The term also encompasses any of the agents approved by a regulatory agency of the US Federal government or listed in the US Pharmacopeia for use in animals, including humans.

The term “pharmaceutical composition” shall mean a composition comprising at least one active ingredient, whereby the composition is amenable to investigation for a specified, efficacious outcome in a mammal (for example, without limitation, a human). Those of ordinary skill in the art will understand and appreciate the techniques appropriate for determining whether an active ingredient has a desired efficacious outcome based upon the needs of the artisan.

As used herein, the term “pharmaceutically-acceptable carrier” means a chemical composition with which an appropriate compound or derivative can be combined and which, following the combination, can be used to administer the appropriate compound to a subject.

As used herein, the term “physiologically acceptable” ester or salt means an ester or salt form of the active ingredient which is compatible with any other ingredients of the pharmaceutical composition, which is not deleterious to the subject to which the composition is to be administered.

“Pharmaceutically acceptable” means physiologically tolerable, for either human or veterinary application.

As used herein, “pharmaceutical compositions” include formulations for human and veterinary use.

“Plurality” means at least two.

A “polynucleotide” means a single strand or parallel and anti-parallel strands of a nucleic acid. Thus, a polynucleotide may be either a single-stranded or a double-stranded nucleic acid.

“Polypeptide” refers to a polymer composed of amino acid residues, related naturally occurring structural variants, and synthetic non-naturally occurring analogs thereof linked via peptide bonds, related naturally occurring structural variants, and synthetic non-naturally occurring analogs thereof.

“Synthetic peptides or polypeptides” means a non-naturally occurring peptide or polypeptide. Synthetic peptides or polypeptides can be synthesized, for example, using an automated polypeptide synthesizer. Various solid phase peptide synthesis methods are known to those of skill in the art.

The term “prevent,” as used herein, means to stop something from happening, or taking advance measures against something possible or probable from happening. In the context of medicine, “prevention” generally refers to action taken to decrease the chance of getting a disease or condition.

A “preventive” or “prophylactic” treatment is a treatment administered to a subject who does not exhibit signs, or exhibits only early signs, of a disease or disorder. A prophylactic or preventative treatment is administered for the purpose of decreasing the risk of developing pathology associated with developing the disease or disorder.

“Primer” refers to a polynucleotide that is capable of specifically hybridizing to a designated polynucleotide template and providing a point of initiation for synthesis of a complementary polynucleotide. Such synthesis occurs when the polynucleotide primer is placed under conditions in which synthesis is induced, i.e., in the presence of nucleotides, a complementary polynucleotide template, and an agent for polymerization such as DNA polymerase. A primer is typically single-stranded, but may be double-stranded. Primers are typically deoxyribonucleic acids, but a wide variety of synthetic and naturally occurring primers are useful for many applications. A primer is complementary to the template to which it is designed to hybridize to serve as a site for the initiation of synthesis, but need not reflect the exact sequence of the template. In such a case, specific hybridization of the primer to the template depends on the stringency of the hybridization conditions. Primers can be labeled with, e.g., chromogenic, radioactive, or fluorescent moieties and used as detectable moieties.

A “prodrug” refers to an agent that is converted into the parent drug in vivo. Prodrugs are often useful because, in some situations, they may be easier to administer than the parent drug. They may, for instance, be bioavailable by oral administration whereas the parent is not. The prodrug may also have improved solubility in pharmaceutical compositions over the parent drug, or may demonstrate increased palatability or be easier to formulate.

As used herein, the term “promoter/regulatory sequence” means a nucleic acid sequence which is required for expression of a gene product operably linked to the promoter/regulator sequence. In some instances, this sequence may be the core promoter sequence and in other instances, this sequence may also include an enhancer sequence and other regulatory elements which are required for expression of the gene product. The promoter/regulatory sequence may, for example, be one which expresses the gene product in a tissue specific manner.

A “constitutive” promoter is a promoter which drives expression of a gene to which it is operably linked, in a constant manner in a cell. By way of example, promoters which drive expression of cellular housekeeping genes are considered to be constitutive promoters.

An “inducible” promoter is a nucleotide sequence which, when operably linked with a polynucleotide which encodes or specifies a gene product, causes the gene product to be produced in a living cell substantially only when an inducer which corresponds to the promoter is present in the cell.

A “tissue-specific” promoter is a nucleotide sequence which, when operably linked with a polynucleotide which encodes or specifies a gene product, causes the gene product to be produced in a living cell substantially only if the cell is a cell of the tissue type corresponding to the promoter.

As used herein, “protecting group” with respect to a terminal amino group refers to a terminal amino group of a peptide, which terminal amino group is coupled with any of various amino-terminal protecting groups traditionally employed in peptide synthesis. Such protecting groups include, for example, acyl protecting groups such as formyl, acetyl, benzoyl, trifluoroacetyl, succinyl, and methoxysuccinyl; aromatic urethane protecting groups such as benzyloxycarbonyl; and aliphatic urethane protecting groups, for example, tert-butoxycarbonyl or adamantyloxycarbonyl. See Gross and Mienhofer, eds., The Peptides, vol. 3, pp. 3-88 (Academic Press, New York, 1981) for suitable protecting groups.

As used herein, “protecting group” with respect to a terminal carboxy group refers to a terminal carboxyl group of a peptide, which terminal carboxyl group is coupled with any of various carboxyl-terminal protecting groups. Such protecting groups include, for example, tert-butyl, benzyl or other acceptable groups linked to the terminal carboxyl group through an ester or ether bond.

The term “protein” typically refers to large polypeptides. Conventional notation is used herein to portray polypeptide sequences: the left-hand end of a polypeptide sequence is the amino-terminus; the right-hand end of a polypeptide sequence is the carboxyl-terminus.

The term “protein regulatory pathway”, as used herein, refers to both the upstream regulatory pathway which regulates a protein, as well as the downstream events which that protein regulates. Such regulation includes, but is not limited to, transcription, translation, levels, activity, posttranslational modification, and function of the protein of interest, as well as the downstream events which the protein regulates.

The terms “protein pathway” and “protein regulatory pathway” are used interchangeably herein.

As used herein, the term “purified” and like terms relate to an enrichment of a molecule or compound relative to other components normally associated with the molecule or compound in a native environment. The term “purified” does not necessarily indicate that complete purity of the particular molecule has been achieved during the process. A “highly purified” compound as used herein refers to a compound that is greater than 90% pure. In particular, purified sperm cell DNA refers to DNA that does not produce significant detectable levels of non-sperm cell DNA upon PCR amplification of the purified sperm cell DNA and subsequent analysis of that amplified DNA. A “significant detectable level” is an amount of contaminate that would be visible in the presented data and would need to be addressed/explained during analysis of the forensic evidence.

“Recombinant polynucleotide” refers to a polynucleotide having sequences that are not naturally joined together. An amplified or assembled recombinant polynucleotide may be included in a suitable vector, and the vector can be used to transform a suitable host cell.

A recombinant polynucleotide may serve a non-coding function (e.g., promoter, origin of replication, ribosome-binding site, etc.) as well.

A host cell that comprises a recombinant polynucleotide is referred to as a “recombinant host cell.” A gene which is expressed in a recombinant host cell wherein the gene comprises a recombinant polynucleotide, produces a “recombinant polypeptide.”

A “recombinant polypeptide” is one which is produced upon expression of a recombinant polynucleotide.

A “receptor” is a compound that specifically binds to a ligand.

A “ligand” is a compound that specifically binds to a target receptor.

The term “regulate” refers to either stimulating or inhibiting a function or activity of interest.

A “sample,” as used herein, refers preferably to a biological sample from a subject, including, but not limited to, normal tissue samples, diseased tissue samples, biopsies, blood, saliva, feces, semen, tears, and urine. A sample can also be any other source of material obtained from a subject which contains cells, tissues, or fluid of interest. A sample can also be obtained from cell or tissue culture. As used herein, “serum” refers to the protein-rich liquid remaining after blood has clotted.

The term “serum half-life” is the term used when describing the amount of time a molecule exists when administered into the blood of a subject and is not limited to “serum”.

By the term “signal sequence” is meant a polynucleotide sequence which encodes a peptide that directs the path a polypeptide takes within a cell, i.e., it directs the cellular processing of a polypeptide in a cell, including, but not limited to, eventual secretion of a polypeptide from a cell. A signal sequence is a sequence of amino acids which are typically, but not exclusively, found at the amino terminus of a polypeptide which targets the synthesis of the polypeptide to the endoplasmic reticulum. In some instances, the signal peptide is proteolytically removed from the polypeptide and is thus absent from the mature protein.

As used herein, “site-specific” refers to incorporation of a certain molecule into another molecule or conjugation of a certain molecule to another molecule at a specific position of the second molecule.

By “small interfering RNAs (siRNAs)” is meant, inter alia, an isolated dsRNA molecule comprised of both a sense and an anti-sense strand. In one aspect, it is greater than 10 nucleotides in length. siRNA also refers to a single transcript which has both the sense and complementary antisense sequences from the target gene, e.g., a hairpin. siRNA further includes any form of dsRNA (proteolytically cleaved products of larger dsRNA, partially purified RNA, essentially pure RNA, synthetic RNA, recombinantly produced RNA) as well as altered RNA that differs from naturally occurring RNA by the addition, deletion, substitution, and/or alteration of one or more nucleotides.

As used herein, the term “solid support” relates to a solvent insoluble substrate that is capable of forming linkages (e.g., covalent bonds) with various compounds. The support can be either biological in nature, such as, without limitation, a cell or bacteriophage particle, or synthetic, such as, without limitation, an acrylamide derivative, agarose, cellulose, nylon, silica, or magnetized particles.

By the term “specifically binds to”, as used herein, is meant when a compound or ligand functions in a binding reaction or assay conditions which is determinative of the presence of the compound in a sample of heterogeneous compounds.

The term “standard,” as used herein, refers to something used for comparison. For example, it can be a known standard agent or compound which is administered and used for comparing results when administering a test compound, or it can be a standard parameter or function which is measured to obtain a control value when measuring an effect of an agent or compound on a parameter or function. Standard can also refer to an “internal standard”, such as an agent or compound which is added at known amounts to a sample and is useful in determining such things as purification or recovery rates when a sample is processed or subjected to purification or extraction procedures before a marker of interest is measured. Internal standards are often a purified marker of interest which has been labeled, such as with a radioactive isotope, allowing it to be distinguished from an endogenous marker.

A “subject” of analysis, diagnosis, or treatment is an animal. Such animals include mammals, preferably a human. Mammals include, for example, humans; non human primates, e.g. apes and monkeys; and non-primates, e.g. dogs, cats, cattle, horses, sheep, and goats. Non mammals include, for example, fish and birds.

As used herein, a “subject in need thereof” is a patient, animal, mammal, or human, who will benefit from the method of this invention.

As used herein, a “substantially homologous amino acid sequences” includes those amino acid sequences which have at least about 95% homology, preferably at least about 96% homology, more preferably at least about 97% homology, even more preferably at least about 98% homology, and most preferably at least about 99% or more homology to an amino acid sequence of a reference antibody chain. Amino acid sequence similarity or identity can be computed by using the BLASTP and TBLASTN programs which employ the BLAST (basic local alignment search tool) 2.0.14 algorithm. The default settings used for these programs are suitable for identifying substantially similar amino acid sequences for purposes of the present invention.

“Substantially homologous nucleic acid sequence” means a nucleic acid sequence corresponding to a reference nucleic acid sequence wherein the corresponding sequence encodes a peptide having substantially the same structure and function as the peptide encoded by the reference nucleic acid sequence; e.g., where only changes in amino acids not significantly affecting the peptide function occur. Preferably, the substantially identical nucleic acid sequence encodes the peptide encoded by the reference nucleic acid sequence. The percentage of identity between the substantially similar nucleic acid sequence and the reference nucleic acid sequence is at least about 50%, 65%, 75%, 85%, 95%, 99% or more. Substantial identity of nucleic acid sequences can be determined by comparing the sequence identity of two sequences, for example by physical/chemical methods (i.e., hybridization) or by sequence alignment via computer algorithm. Suitable nucleic acid hybridization conditions to determine if a nucleotide sequence is substantially similar to a reference nucleotide sequence are: 7% sodium dodecyl sulfate SDS, 0.5 M NaPO₄, 1 mM EDTA at 50° C. with washing in 2× standard saline citrate (SSC), 0.1% SDS at 50° C.; preferably in 7% (SDS), 0.5 M NaPO₄, 1 mM EDTA at 50° C. with washing in 1×SSC, 0.1% SDS at 50° C.; preferably 7% SDS, 0.5 M NaPO₄, 1 mM EDTA at 50° C. with washing in 0.5×SSC, 0.1% SDS at 50° C.; and more preferably in 7% SDS, 0.5 M NaPO₄, 1 mM EDTA at 50° C. with washing in 0.1×SSC, 0.1% SDS at 65° C. Suitable computer algorithms to determine substantial similarity between two nucleic acid sequences include, GCS program package (Devereux et al., 1984 Nucl. Acids Res. 12:387), and the BLASTN or FASTA programs (Altschul et al., 1990 Proc. Natl. Acad. Sci. USA. 1990 87:14:5509-13; Altschul et al., J. Mol. Biol. 1990 215:3:403-10; Altschul et al., 1997 Nucleic Acids Res. 25:3389-3402). The default settings provided with these programs are suitable for determining substantial similarity of nucleic acid sequences for purposes of the present invention.

The term “substantially pure” describes a compound, e.g., a protein or polypeptide which has been separated from components which naturally accompany it. Typically, a compound is substantially pure when at least 10%, more preferably at least 20%, more preferably at least 50%, more preferably at least 60%, more preferably at least 75%, more preferably at least 90%, and most preferably at least 99% of the total material (by volume, by wet or dry weight, or by mole percent or mole fraction) in a sample is the compound of interest. Purity can be measured by any appropriate method, e.g., in the case of polypeptides by column chromatography, gel electrophoresis, or HPLC analysis. A compound, e.g., a protein, is also substantially purified when it is essentially free of naturally associated components or when it is separated from the native contaminants which accompany it in its natural state.

The term “symptom,” as used herein, refers to any morbid phenomenon or departure from the normal in structure, function, or sensation, experienced by the patient and indicative of disease. In contrast, a “sign” is objective evidence of disease. For example, a bloody nose is a sign. It is evident to the patient, doctor, nurse and other observers.

A “therapeutic” treatment is a treatment administered to a subject who exhibits signs of pathology for the purpose of diminishing or eliminating those signs.

A “therapeutically effective amount” of a compound is that amount of compound which is sufficient to provide a beneficial effect to the subject to which the compound is administered.

The term to “treat,” as used herein, means reducing the frequency with which symptoms are experienced by a patient or subject or administering an agent or compound to reduce the frequency with which symptoms are experienced.

As used herein, the term “treating” includes prophylaxis of the specific disorder or condition, or alleviation of the symptoms associated with a specific disorder or condition and/or preventing or eliminating said symptoms.

A “prophylactic” treatment is a treatment administered to a subject who does not exhibit signs of a disease or exhibits only early signs of the disease for the purpose of decreasing the risk of developing pathology associated with the disease.

### Disease/Disorder/Injury

Hyperglycemia, or high blood sugar (also spelled hyperglycaemia or hyperglycemia) is a condition in which an excessive amount of glucose circulates in the blood plasma. This is generally a blood sugar level higher than 11.1 mmol/l (200 mg/dl), but symptoms may not start to become noticeable until even higher values such as 15-20 mmol/l (˜250-300 mg/dl). A subject with a consistent range between ˜5.6 and ˜7 mmol/l (100-126 mg/dl) (American Diabetes Association guidelines) is considered hyperglycemic, while above 7 mmol/l (126 mg/dl) is generally held to have diabetes. Chronic levels exceeding 7 mmol/l (125 mg/dl) can produce organ damage.

Temporary hyperglycemia is often benign and asymptomatic. Blood glucose levels can rise well above normal for significant periods without producing any permanent effects or symptoms. However, chronic hyperglycemia at levels more than slightly above normal can produce a very wide variety of serious complications, including kidney damage, neurological damage, cardiovascular damage, damage to the retina or damage to feet and legs. Diabetic neuropathy may be a result of long-term hyperglycemia.

Acute hyperglycemia involving glucose levels that are extremely high is a medical emergency and can rapidly produce serious complications (such as fluid loss through osmotic diuresis). The following symptoms may be associated with acute or chronic hyperglycemia: Polyphagia—frequent hunger, especially pronounced hunger; Polydipsia—frequent thirst, especially excessive thirst; Polyuria —increased volume of urination (not an increased frequency for urination); Blurred vision; Fatigue; Weight loss; Poor wound healing (cuts, scrapes, etc.); Dry mouth; Dry or itchy skin; Tingling in feet or heels; Erectile dysfunction; Recurrent infections, external ear infections (swimmer's ear); Cardiac arrhythmia; Stupor; Coma; and/or Seizures.

Signs and symptoms of diabetic ketoacidosis may include: Ketoacidosis; Kussmaul hyperventilation: deep, rapid breathing; Confusion or a decreased level of consciousness; Dehydration due to glycosuria and osmotic diuresis; Acute hunger and/or thirst; ‘Fruity’ smelling breath odor; and/or Impairment of cognitive function, along with increased sadness and anxiety.

Hyperglycemia can be a serious problem if not treated in time. In untreated hyperglycemia, a condition called ketoacidosis (contrast ketosis) could occur. Ketoacidosis develops when the body does not have enough insulin. Without insulin, the body isn't able to utilize the glucose for fuel, so the body starts to break down fats for energy.

Ketoacidosis is a life-threatening condition which needs immediate treatment. Symptoms include: shortness of breath, breath that smells fruity (such as pear drops), nausea and vomiting, and very dry mouth. Chronic hyperglycemia (high blood sugar) injures the heart in patients without a history of heart disease or diabetes and is strongly associated with heart attacks and death in subjects with no coronary heart disease or history of heart failure.

A high proportion of patients suffering an acute stress such as stroke or myocardial infarction may develop hyperglycemia, even in the absence of a diagnosis of diabetes. (Or perhaps stroke or myocardial infarction was caused by hyperglycemia and undiagnosed diabetes.) Human and animal studies suggest that this is not benign, and that stress-induced hyperglycemia is associated with a high risk of mortality after both stroke and myocardial infarction.

The following conditions can also cause hyperglycemia in the absence of diabetes. 1) Dysfunction of the thyroid, adrenal, and pituitary glands 2) Numerous diseases of the pancreas 3) Severe increases in blood glucose may be seen in sepsis and certain infections 4) Intracranial diseases (frequently overlooked) can also cause hyperglycemia. Encephalitis, brain tumors (especially those located near the pituitary gland), brain bleeds, and meningitis are prime examples. 5) Mild to high blood sugar levels are often seen in convulsions and terminal stages of many diseases. Surgeries can (temporarily) increase glucose levels. Stress (e.g., from surgery) and physical trauma can increase levels. Hyperglycemia is a common occurrence following trauma or surgery, even in patients without a previous history of diabetes.

### Agents/Compounds

ATGL plays a role in efficient mobilization of triglyceride (TG) stores in adipose tissue and non-adipose tissues. Therefore, ATGL plays a role in the determination of the availability of fatty acids for metabolic reactions. ATGL activity is regulated by a complex network of hormones which control enzyme expression and the interaction of the enzyme with regulatory proteins; for instance, ATGL activity is regulated by a complex network of lipolytic and anti-lipolytic hormones. ATGL is stimulated by the presence of an activator protein as observed for other TG lipases, such as pancreatic lipase or lipoprotein lipase.

The present invention provides for inhibiting adipose tissue lipolysis by inhibiting ATGL activity, synthesis, levels and any upstream or downstream component of its pathway. In one aspect, an inhibitor selected from the group consisting of inhibitors of activity, expression, etc., as well as stimulators of its degradation.

Accordingly, in various embodiments, the invention can provide a compound of formula (I):

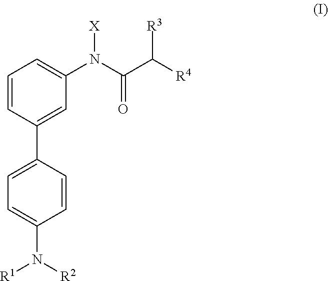

wherein R¹, R², R³, R⁴ and X are individually selected from H, (C₁-C₆) alkyl, phenyl, benzyl, phenylethyl or a pharmaceutically acceptable slat thereof.

Another embodiment provides a compound of formula (II):

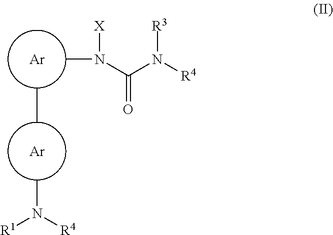

wherein R¹, R², R³, R⁴ and X are individually H, (C₁-C₆) alkyl, (C₃-C₆)cycloalkyl, (C₁-C₆)alkanoyl, trifluoromethyl, hydroxy (C₁-C₆)alkyl, aryl, aryl(C₁-C₆)alkyl;
each Ar is individually selected from aryl; or a pharmaceutically acceptable salt thereof.

A further embodiment provides a compound of formula (III):

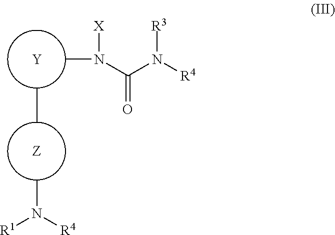

wherein R¹, R², R³, R⁴ and X are individually H, (C₁-C₆) alkyl, (C₃-C₆)cycloalkyl, (C₁-C₆)alkanoyl, trifluoromethyl, hydroxy (C₁-C₆)alkyl, aryl, aryl(C₁-C₆)alkyl;
Y and Z are selected from aryl or Het, such as heteroaryl and wherein Y and Z comprise a bond connecting two carbon atoms of Y and Z; or a pharmaceutically acceptable salt thereof.

In one embodiment, a useful compound of the invention is Atglistatin (N′-[4′-(dimethylamino) [1,1′-biphenyl]-3-yl]-N,N-dimethyl-urea). Its CAS number is 1469924-27-3. It has the following structure:

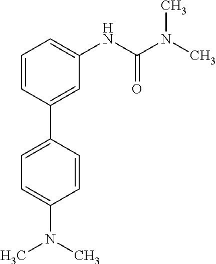 [SMILES: CN(C)C(=O)Nc1cccc(-c2ccc(N(C)C)cc2)c1]

Adipose triglyceride lipase (ATGL or PNPLA2) catalyzes the initial step in triglyceride hydrolysis in adipocyte and non-adipocyte lipid droplets, generating diacylglycerol (rate limiting enzyme involved in the mobilization of fatty acids from cellular triglyceride stores). ATGL generates diacylglycerol from cellular triglyceride stores, which is then degraded by hormone-sensitive lipase (HSL) and monoglyceride lipase into glycerol and fatty acids, promoting the synthesis of lipotoxic metabolites that have been associated with the development of insulin resistance.

Atglistatin is a potent, selective, and competitive inhibitor of ATGL (IC₅₀=0.7 μM). It does not inhibit hormone-sensitive lipase, monoglyceride lipase, pancreatic lipase, or lipoprotein lipase PNPLA6 and PNPLA7. Atglistatin blocks lipolysis (to reduce fatty acid mobilization) by ATGL in vitro, in white adipose tissue organ cultures, and in vivo.

The present invention further encompasses the use of long-chain acyl-CoAs to inhibit ATGL activity, levels, etc. to regulate hyperglycemia and insulin resistance (see Nagy et al., Biochim Biophys Acta. 2014 Apr. 4; 1841(4):588-94. doi: 10.1016/j.bbalip.2014.01.005. Epub 2014 Jan. 16, who demonstrated that adipose triglyceride lipase activity is inhibited by long-chain acyl-coenzyme A).

The present invention also encompasses the of hormone-sensitive lipases, such as those disclosed in Lowe et al. Bioorg. & Med. Chem Letts. (2004) 14:3155-3159. These inhibitors include, but are not limited to:

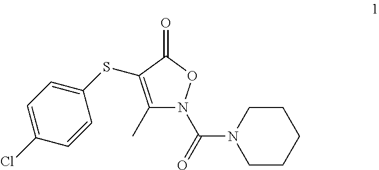 [SMILES: Cc1c(Sc2ccc(Cl)cc2)c(=O)on1C(=O)N1CCCCC1]

as well as derivatives thereof including those compounds listed in the Tables 1-4 below:

TABLE 1
IC₅₀ values (in nM) for 4-substituted 5-(2H)-isoxazolones

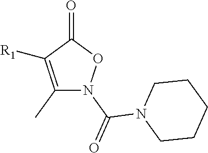

| | | IC₅₀ (HSL) | IC₅₀ (3T3- |
|---|---|---|---|
| Compound | R₁ | (nM)^{a} | L1) (nM)^{a} |
| 1 | S4-Chlorophenyl | 6 | 135 |
| 2 | SO₂(4-chloro)phenyl | 35 | 1270 |
| 3 | S Phenyl | 6 | 190 |
| 4 | Benzyl | 5 | 90 |
| 5 | CH₃ | 13 | 655 |
| 6 | Ethyl | 18 | 325 |
| 7 | n-Butyl | 7 | 61 |
| 8 | Isopropyl | 2 | 75 |
| 9 | Isobutyl | 5 | 32 |
| 10 | tert-Butyl | 14 | 90 |
| 11 | Cyclopentyl | 6 | 22 |
| 12 | Cyclohexyl | 11 | 27 |
| 13 | 4-Tetrahydropyranyl | 71 | 200 |
| 14 | 4-Tetrahydrothiopyranyl | 11 | 98 |
| 15 | 4-Tetrahydrothiopyranyl | 55 | 380 |
| | dioxide | | |
| 16 | CH₂CH₂OCH₃ | 10 | 180 |
| 17 | CH₂CH₂OPh | 4 | 2 |
| 18 | CH₂(N—CH₃)-2-indolyl | 17 | 177 |
| 19 | Phenyl | 17 | 150 |

^{a}Values are means of three experiments.

TABLE 2
IC₅₀ values (in nM) for acyclic ureas

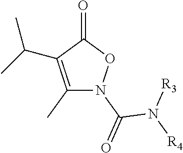

| | | | IC₅₀ (HSL) | IC₅₀ (3T3- |
|---|---|---|---|---|
| Compound | R₃ | R₄ | (nM)^{a} | L1) (nM)^{a} |
| 20 | H | tert-Butyl | >1000 | Na |
| 21 | H | Phenyl | >1000 | Na |
| 22 | H | 8-Quinolinyl | Na | Na |
| 23 | H | CH₂cyclohexyl | 72 | Na |
| 24 | CH₃ | Cyclohexyl | 9 | >1000 |
| 25 | CH₃ | Benzyl | 5 | 67 |
| 26 | CH₃ | (2-Fluoro)benzyl | 4 | 10 |
| 27 | CH₃ | (3-Fluoro)benzyl | 5 | 12 |
| 28 | CH₃ | (4-Fluoro)benzyl | 111 | 1000 |
| 29 | CH₃ | (4-Methyl)benzyl | 408 | >1000 |
| 30 | CH₃ | CH₂(2-furanyl) | 4 | 9 |
| 31 | CH₃ | CH₂(2-thienyl) | 2 | 3 |
| 32 | CH₃ | CH₂CH₂CN | >1000 | Na |
| 33 | CH₃ | CH₂CH₂Ph | 180 | 300 |
| 34 | CH₃ | CH₂CH₂(2-indolyl) | Na | Na |
| 35 | CH₃ | CH₂CONH₂ | >1000 | Na |
| 36 | CH₃ | Phenyl | 7 | 190 |
| 37 | CH₃ | 4-Chlorophenyl | 17 | >1000 |
| 38 | CH₃ | 2-Pyridyl | 240 | Na |
| 39 | Ethyl | Ethyl | 1400 | 600 |
| 40 | Isopropyl | Isopropyl | Na | Na |

^{a}Values are means of three experiments (Na = not active).

TABLE 3
IC₅₀ values (in nM) for cyclic amine-derived ureas

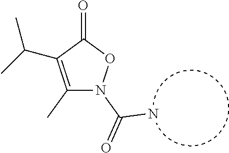

| | | IC₅₀ (HSL) | IC₅₀ (3T3- |
|---|---|---|---|
| Compound | Cyclic amine | (nM)^{a} | L1) (nM)^{a} |
| 8 | Piperidine | 2 | 75 |
| 41 | Pyrrolidine | 15 | 28 |
| 42 | 2,5-Dimethylpyrrolidine | Na | Na |
| 43 | 3-Pyrroline | 72 | 120 |
| 44 | Indoline | >1000 | Na |
| 45 | Tetrahydroquinoline | 60 | >1000 |
| 46 | Piperazine | >1000 | Na |
| 47 | N-Methylpiperazine | 314 | >1000 |
| 48 | N-Benzylpiperazine | 65 | 56 |
| 49 | N-Penylpiperazine | 6 | 27 |
| 50 | N-(2-Chlorophenyl) | 30 | 31 |
| | piperazine | | |
| 51 | N-(3-Methoxyphenyl) | 1 | 13 |
| | piperazine | | |
| 52 | N-(2-Pyrimidinyl) | 67 | 51 |
| | piperazine | | |
| 53 | Morpholine | 52 | >1000 |
| 54 | Thiomorpholine | 8 | 290 |
| 55 | Homopiperidine | 62 | 215 |
| 56 | Azacyclooctane | 900 | Na |

^{a}Values are means of three experiments (Na = not active).

IC₅₀ values (in nM) for piperidine derivatives

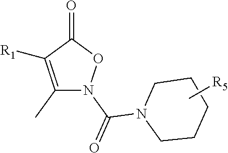

| Compound | R₁ | R₅ | IC₅₀ (HSL) (nM)^{a} | IC₅₀ (3T3-L1) (nM)^{a} |
|---|---|---|---|---|
| 8 | Isopropyl | H | 2 | 75 |
| 57 | Isopropyl | 2-Methyl | 93 | 400 |
| 58 | Isopropyl | 3-(R)-Methyl | 20 | 187 |
| 59 | Isopropyl | 3-(S)-Methyl | 3 | 28 |
| 60 | Isopropyl | 3-Hydroxymethyl | 180 | 800 |
| 61 | Isopropyl | 3-CON(Et)₂ | >1000 | Na |
| 62 | Isopropyl | 3-CO₂Et | 28 | 170 |
| 63 | Isopropyl | 4-Methyl | 6 | 40 |
| 64 | n-Butyl | 4-Methyl | 1 | 10 |
| 65 | Isobutyl | 4-Methyl | 3 | 18 |
| 66 | Cyclohexyl | 4-Methyl | 4 | 20 |
| 67 | CH₂CH₂OEt | 4-Methyl | 3 | 20 |
| 68 | 4-Tetrahydrothiopyranyl | 4-Methyl | 6 | 24 |
| 69 | Isopropyl | 4-Benzyl | 102 | >1000 |
| 70 | Isopropyl | 4-Phenyl | 12 | 33 |
| 71 | Isobutyl | 4-Phenyl | 10 | 4 |
| 72 | Cyclopently | 4-Phenyl | 24 | 8 |
| 73 | CH₂CH₂OCH₃ | 4-Phenyl | 11 | 5 |
| 74 | Isopropyl | 4-(3-Fluorophenyl) | 12 | 4 |
| 75 | CH₂CH₂OCH₃ | 4-(3-Fluorophenyl) | 20 | 0.2 |
| 76 | CH₂CH₂OCH₃ | 4-(3-Methylphenyl) | 23 | 4 |
| 77 | Isopropyl | 4-(4-Methylphenyl) | 37 | 246 |
| 78 | Isopropyl | 4-(4-CF₃-phenyl) | 160 | — |
| 79 | Isopropyl | 4-(4-t-Bu-phenyl) | 480 | — |
| 80 | Isopropyl | 4-COphenyl | 250 | >1000 |
| 81 | Isopropyl | 4-CO₂Et | 6 | 49 |
| 82 | Isopropyl | 4-Hydroxy | 166 | 450 |
| 83 | Isopropyl | 4-Keto | 200 | 150 |
| 84 | Isopropyl | 4-CON(CH₃)₂ | >1000 | Na |
| 85 | Isopropyl | isoxazoyl) | 28 | 73 |
| 86 | Isopropyl | 4-(5-CH₃-2-oxadiazolyl) | >1000 | Na |
| 87 | Isopropyl | 4-N-Piperidinyl | Na | Na |
| 88 | Isopropyl | 2,5-Dimethyl | 460 | >1000 |
| 89 | Isopropyl | 3,3-Dimethyl | 7 | 48 |
| 90 | Isopropyl | 3,5-Dimethyl | 326 | 140 |
| 91 | Isopropyl | 4-CN-4-Phenyl | >1000 | Na |
| 92 | Isopropyl | 4-OH-4-Phenyl | >1000 | >1000 |

^{a}Values are means of three experiments (Na = not active).

In one embodiment, inhibitors of Sirtuin 1 such as Sirt 1 siRNA and nicotinamide (NAM) are encompassed by the invention for use in inhibiting the expression, levels, or activity of ATGL.

The present invention further encompasses analogues and derivatives of Atglistatin exhibiting the activity of Atglistatin described herein. Such compounds include, but are not limited to, for example those found in Mayer et al., 2013, Nat. Chem. Biol. 9:12 and in Mayer et al., 2015, Bioorganic & Medicinal Chemistry, 23:2904, as well as pharmaceutically acceptable salts, solvates, or prodrugs thereof. These compounds included:

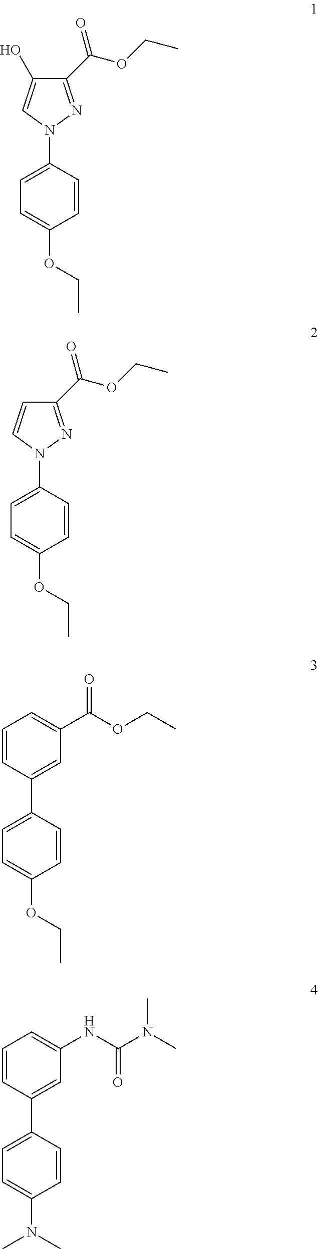

Other useful compounds of the invention include compounds available from ChemDiv or Asinex. These include, but are not limited to ChemDiv 4112-0423 (0875-0003-7092), 4275-2216 (0875-0003-7306), 4356-2701 (0875-0003-7401), 4427-1195 (0875-0003-7446), 3254-0350 (0875-0003-6659), 8012-8567 (0875-0003-7167), and 4209-0100 (0875-0003-7242) and Asinex AEM 11160086 (0876-0001-1449), as well as pharmaceutically acceptable salts, solvates, or prodrugs thereof (see U.S. Pat. No. 8,993,509; columns 49-51)—see compounds below:

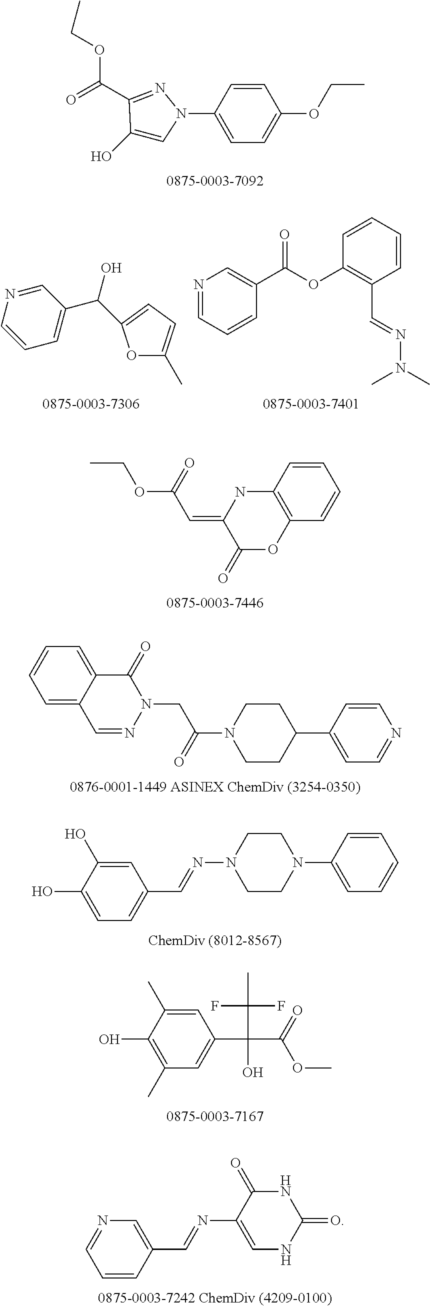

Another useful compound for use in the invention is GS-9667, also known as CVT-3619. Its chemical name is 5′-S-(2-Fluorophenyl)-N-[(1R,2R)-2-hydroxycyclopentyl]-5′-thioadenosine. It has the structure:

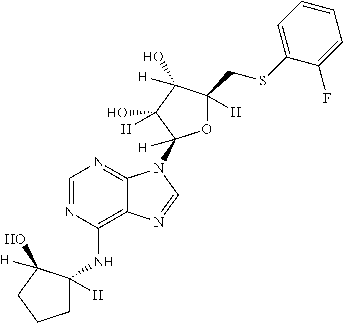 [SMILES: O[C@@H]1[C@H](O)[C@@H](CSc2ccccc2F)O[C@H]1n1cnc2c(N[C@@H]3CCC[C@H]3O)ncnc21]

or pharmaceutically acceptable salts thereof.

Also included is a compound of the formula:

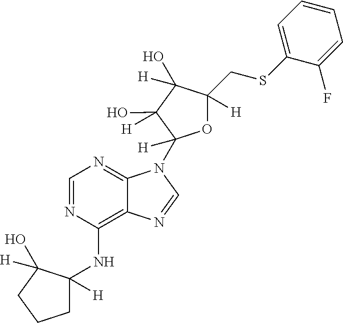 [SMILES: OC1CCCC1Nc1ncnc2c1ncn2C1OC(CSc2ccccc2F)C(O)C1O]

or a pharmaceutically acceptable salt thereof.

In one embodiment, the compound is a compound of the formula:

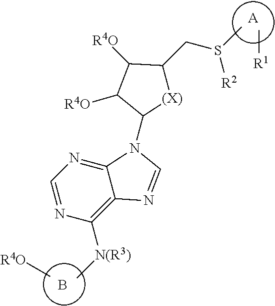

wherein A is aryl, R′ is adjacent to (S—) and is halo, R² is absent or is (O), X is —(CH₂)—, O or S, R³ is H, phenyl, (C₁-C₆)alkyl or phenethyl, R⁴ is each individually H, (C₁-C₆)alkyl, (C₁-C₆)alkenyl, (C₁-C₆)alkynyl, B is cycloalkyl, wherein —OR⁴ is adjacent to —N, or a pharmaceutically acceptable salt of said compound.

In one embodiment, the compound is a compound of the formula:

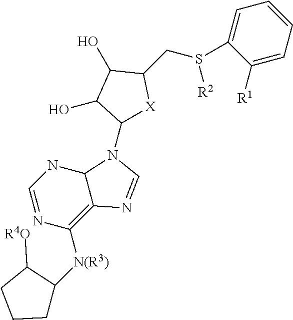

wherein R¹ is halo, R² is absent or is (O), X is O, S or —(CH₂)—, R³ is H, (C₁-C₆)alkyl, phenyl, benzyl or phenethyl, R⁴ is H, (C₁-C₆)alkyl, (C₁-C₆) alkenyl or (C₁-C₆)alkynyl, or a pharmaceutically acceptable salt of said compound.

Presented here are unexpected results from inhibiting ATGL activity. Therefore, the present invention encompasses compositions and methods useful for eliciting the results disclosed herein using other molecules that inhibit or reduce ATGL activity, expression, levels, etc. Such regulation can also occur upstream or downstream and the inhibitors of the invention need not act directly on ATGL.

### Chemical Definitions

The following definitions are used, unless otherwise described: halo is fluoro, chloro, bromo, or iodo.

The term “haloalkyl” as used herein refers to an alkyl radical bearing at least one halogen substituent, for example, chloromethyl, fluoroethyl or trifluoromethyl and the like.

Alkyl, alkoxy, etc. denote both straight and branched groups; but reference to an individual radical such as “propyl” embraces only the straight chain radical, a branched chain isomer such as “isopropyl” being specifically referred to.

The term “C₁-Cₙ alkyl” wherein n is an integer, as used herein, represents a branched or linear alkyl group having from one to the specified number of carbon atoms. Typically, C₁-C₆ alkyl groups include, but are not limited to, methyl, ethyl, n-propyl, iso-propyl, butyl, iso-butyl, sec-butyl, tert-butyl, pentyl, hexyl, and the like.

The term “C₂-Cₙ alkenyl” wherein n is an integer, as used herein, represents an olefinically unsaturated branched or linear group having from 2 to the specified number of carbon atoms and at least one double bond. Examples of such groups include, but are not limited to, 1-propenyl, 2-propenyl, 1,3-butadienyl, 1-butenyl, hexenyl, pentenyl, and the like.

The term “C₂-Cₙ alkynyl” wherein n is an integer refers to an unsaturated branched or linear group having from 2 to the specified number of carbon atoms and at least one triple bond. Examples of such groups include, but are not limited to, 1-propynyl, 2-propynyl, 1-butynyl, 2-butynyl, 1-pentynyl, and the like.

The term “C₃-Cₙ cycloalkyl” wherein n=8, represents cyclopropyl, cyclobutyl, cyclopentyl, cyclohexyl, cycloheptyl, and cyclooctyl; (C₃-C₆)cycloalkyl(C₁-C₆)alkyl can be cyclopropylmethyl, cyclobutylmethyl, cyclopentylmethyl, cyclohexylmethyl, 2-cyclopropylethyl, 2-cyclobutylethyl, 2-cyclopentylethyl, or 2-cyclohexylethyl.

(C₁-C₆)alkanoyl can be acetyl, propanoyl or butanoyl; hydroxy(C₁-C₅)alkyl can be hydroxymethyl, 1-hydroxyethyl, 2-hydroxyethyl, 1-hydroxypropyl, 2-hydroxypropyl, 3-hydroxypropyl, 1-hydroxybutyl, 4-hydroxybutyl, 1-hydroxypentyl, 5-hydroxypentyl, 1-hydroxyhexyl, or 6-hydroxyhexyl;

(C₂-C₆)alkanoyloxy can be acetoxy, propanoyloxy, butanoyloxy, isobutanoyloxy, pentanoyloxy, or hexanoyloxy;

Wherein any (C₁-C₆)alkyl of R1, R2, R3, or R4 is optionally substituted with one or more (e.g. 1, 2, 3, or 4) halo, hydroxy, (C₁-C₆)alkoxy, (C₃-C₆)cycloalkyloxy, (C₁-C₆)alkanoyl, (C₁-C₆)alkanoyloxy, trifluoromethyl, azido, cyano, oxo (═O), (C₁-C₆)alkyl, (C₃-C₆)cycloalkyl, (C₃-C₆)cycloalkyl(C₁-C₆)alkyl, (C₁-C₆)alkyl-S(C₁-C₆)alkyl-, aryl, Het, aryl(C₁-C₆)alkyl, or Het (C₁-C₆)alkyl, or X; wherein each Rₐⱼ and Rₐₖ is independently hydrogen, (C₁-C₆)alkyl, (C₃-C₆)cycloalkyl, phenyl, benzyl, or phenethyl.

As used herein, the term “optionally substituted” typically refers to from zero to four substituents, wherein the substituents are each independently selected. Each of the independently selected substituents may be the same or different than other substituents. For example, the substituents of an R group of a formula may be optionally substituted (e.g., from 1 to 4 times) with independently selected H, halogen, hydroxy, acyl, alkyl, alkenyl, alkynyl, cycloalkyl, heterocyclo, aryl, heteroaryl, alkoxy, amino, amide, thiol, sulfone, sulfoxide, oxo, oxy, nitro, carbonyl, carboxy, amino acid sidechain and amino acid.

As used herein the term “aryl” refers to an optionally substituted mono- or bicyclic carbocyclic ring system having one or two aromatic rings including, but not limited to, phenyl, benzyl, naphthyl, tetrahydronaphthyl, indanyl, indenyl, and the like; and heteroaryl can be furyl, imidazolyl, triazolyl, triazinyl, oxazoyl, isoxazoyl, thiazolyl, isothiazoyl, pyrazolyl, pyrrolyl, pyrazinyl, tetrazolyl, pyridyl, (or its N-oxide), thienyl, pyrimidinyl (or its N-oxide), indolyl, isoquinolyl (or its N-oxide) or quinolyl (or its N-oxide). Optionally substituted aryl includes aryl compounds having from zero to four substituents, and substituted aryl includes aryl compounds having one or more substituents. The term (C₅-C₈ alkyl)aryl refers to any aryl group which is attached to the parent moiety via the alkyl group.

Aryl denotes a phenyl radical or an ortho-fused bicyclic carbocyclic radical having about nine to ten ring atoms in which at least one ring is aromatic. Het or Het¹ is (C₁-C₆)alkyl, (C₃-C₆)cycloalkyl or is a radical of a monocyclic, bicyclic, or tricyclic ring system containing a total of 3-20 atoms, including one or more (e.g., 1, 2, 3, 4, 5, 6, or 7) carbon atoms, and one or more (e.g., 1, 2, 3, 4, or 5) heteroatoms selected from oxygen, sulfur, and N(X) wherein X is absent or is H, O, (C₁-C₄)alkyl, phenyl or benzyl, wherein one or more ring carbons of Het can optionally be substituted with oxo (═O).

Any aryl, Ar, or Het may optionally be substituted with one or 0.10 more substituents selected from the group consisting of halo, hydroxy, (C₁-C₆)alkyl, (C₃-C₆)cycloalkyl, (C₁-C₆)alkoxy, (C₃-C₆)cycloalkyloxy, (C₁-C₆)alkanoyl, (C₁-C₆)alkanoyloxy, trifluoromethyl, trifluoromethoxy, nitro, cyano, and amino.

“Heterocycle” refers to any stable 4, 5, 6, 7, 8, 9, 10, 11, or 12 membered, (unless the number of members is otherwise recited), monocyclic, bicyclic, or tricyclic heterocyclic ring that is saturated or partially unsaturated, and which consists of carbon atoms and 1, 2, 3, or 4 heteroatoms independently selected from the group consisting of N, O, and S. If the heterocycle is defined by the number of carbons atoms, then from 1, 2, 3, or 4 of the listed carbon atoms are replaced by a heteroatom. If the heterocycle is bicyclic or tricyclic, then at least one of the two or three rings must contain a heteroatom, though both or all three may each contain one or more heteroatoms. The N group may be N, NH, or N-substituent, depending on the chosen ring and if substituents are recited. The nitrogen and sulfur heteroatoms optionally may be oxidized (e.g., S, S(O), S(O)₂, and N—O). The heterocycle may be attached to its pendant group at any heteroatom or carbon atom that results in a stable structure. The heterocycles described herein may be substituted on carbon or on a nitrogen atom if the resulting compound is stable.

“Heteroaryl” refers to any stable 5, 6, 7, 8, 9, 10, 11, or 12 membered, (unless the number of members is otherwise recited), monocyclic, bicyclic, or tricyclic heterocyclic ring that is aromatic, and which consists of carbon atoms and 1, 2, 3, or 4 heteroatoms independently selected from the group consisting of N, O, and S. If the heteroaryl is defined by the number of carbons atoms, then 1, 2, 3, or 4 of the listed carbon atoms are replaced by a heteroatom. If the heteroaryl group is bicyclic or tricyclic, then at least one of the two or three rings must contain a heteroatom, though both or all three may each contain one or more heteroatoms. If the heteroaryl group is bicyclic or tricyclic, then only one of the rings must be aromatic. The N group may be N, NH, or N-substituent, depending on the chosen ring and if substituents are recited. The nitrogen and sulfur heteroatoms may optionally be oxidized (e.g., S, S(O), S(O)₂, and N—O). The heteroaryl ring may be attached to its pendant group at any heteroatom or carbon atom that results in a stable structure. The heteroaryl rings described herein may be substituted on carbon or on a nitrogen atom if the resulting compound is stable.”

The term “heteroatom” means for example oxygen, sulfur, nitrogen, phosphorus, or silicon (including, any oxidized form of nitrogen, sulfur, phosphorus, or silicon; the quaternized form of any basic nitrogen or; a substitutable nitrogen of a heterocyclic ring.

The term “bicyclic” represents either an unsaturated or saturated stable 7- to 12-membered bridged or fused bicyclic carbon ring. The bicyclic ring may be attached at any carbon atom which affords a stable structure. The term includes, but is not limited to, naphthyl, dicyclohexyl, dicyclohexenyl, and the like.

The compounds of the present invention may exist in tautomeric forms and the invention includes both mixtures and separate individual tautomers. For example the following structure:

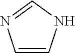

is understood to represent a mixture of the structures:

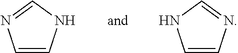 [SMILES: c1c[nH]cn1 ; c1c[nH]cn1]

The term “pharmaceutically-acceptable salt” refers to salts which retain the biological effectiveness and properties of the compounds of the present invention and which are not biologically or otherwise undesirable. In many cases, the compounds of the present invention are capable of forming acid and/or base salts by virtue of the presence of amino and/or carboxyl groups or groups similar thereto. In cases where compounds are sufficiently basic or acidic to form stable nontoxic acid or base salts, administration of the compounds as salts may be appropriate. Examples of pharmaceutically acceptable salts are organic acid addition salts formed with acids which form a physiological acceptable anion, for example, tosylate, methanesulfonate, acetate, citrate, malonate, tartarate, succinate, benzoate, ascorbate, α-ketoglutarate, and α-glycerophosphate. Suitable inorganic salts may also be formed, including hydrochloride, sulfate, nitrate, bicarbonate, and carbonate salts.

Pharmaceutically acceptable salts may be obtained using standard procedures well known in the art, for example by reacting a sufficiently basic compound such as an amine with a suitable acid affording a physiologically acceptable anion. Alkali metal (for example, sodium, potassium or lithium) or alkaline earth metal (for example calcium) salts of carboxylic acids can also be made, as well as ammonium salts or quats.

Compounds of the present invention that have one or more asymmetric carbon atoms may exist as the optically pure enantiomers, or optically pure diastereomers, as well as mixtures of enantiomers, mixtures of diastereomers, and racemic mixtures of such stereoisomers. The present invention includes within its scope all such isomers and mixtures thereof. The compounds of the present invention contain one or more asymmetric centers in the molecule. In accordance with the present invention a structure that does not designate the stereochemistry is to be understood as embracing all the various optical isomers, as well as racemic mixtures thereof. It will be appreciated by those skilled in the art that compounds of the invention having a chiral center may exist in and be isolated in optically active and racemic forms. Some compounds may exhibit polymorphism. It is to be understood that the present invention encompasses any racemic, optically-active, polymorphic, or stereoisomeric form, or mixtures thereof of a compound of the invention, which possess the useful properties described herein, it being well known in the art how to prepare optically active forms (for example, by resolution of the racemic form by recrystallization techniques, by synthesis from optically-active starting materials, by chiral synthesis, or by chromatographic separation using a chiral stationary phase) and how to determine modulatory activity using the standard tests described herein, or using other similar tests which are well known in the art.

A compound of the invention can be administered to a subject in need thereof in an amount that is therapeutically effective for the disease, disorder, or condition to be treated. For example, the dose(s) of Atglistatin or other compounds of the invention to be used can be determined by one of skill in the art and can be based on factors such as the age, weight, sex, and health of the subject, the type of stress being placed upon the subject, etc. In one embodiment, it can be administered at a range from about 0.1 mg/kg body weight to about 100 mg/kg body weight. In one aspect, it can be administered at a range from about 0.5 to about 50. In another aspect, it can be administered at a range from about 1.0 to about 10. It will be appreciated that more than one dose can be administered or that a dose can be split into two or more units for use in a day for example. A compound of the invention can be administered, for example, once a day, once a week, etc. It can also be administered at least twice a day, or at least twice a day, etc. Doses can also be administered in a perioperative setting, for example, before, during, or after surgery.

### Pharmaceutical Compositions and Methods of Treatment

The compounds of the invention can be formulated as pharmaceutical compositions and administered to a mammalian host, such as a human patient in a variety of forms adapted to the chosen route of administration, i.e., orally or parenterally, by intravenous, intramuscular, topical or subcutaneous routes.

In various embodiments, the invention provides pharmaceutical compositions comprising a compound of the invention and a pharmaceutically acceptable excipient.

Another aspect of an embodiment of the invention provides compositions of the compounds of the invention, alone or in combination with another medicament. As set forth herein, compounds of the invention include stereoisomers, tautomers, solvates, prodrugs, pharmaceutically acceptable salts and mixtures thereof. Compositions containing a compound of the invention can be prepared by conventional techniques, e.g. as described in Remington: The Science and Practice of Pharmacy, 19th Ed., 1995, or later versions thereof, incorporated by reference herein. The compositions can appear in conventional forms, for example capsules, tablets, aerosols, solutions, suspensions or topical applications.

Typical compositions include a compound of the invention and a pharmaceutically acceptable excipient which can be a carrier or a diluent. For example, the active compound will usually be mixed with a carrier, or diluted by a carrier, or enclosed within a carrier which can be in the form of an ampoule, capsule, sachet, paper, or other container. When the active compound is mixed with a carrier, or when the carrier serves as a diluent, it can be solid, semi-solid, or liquid material that acts as a vehicle, excipient, or medium for the active compound. The active compound can be adsorbed on a granular solid carrier, for example contained in a sachet. Some examples of suitable carriers are water, salt solutions, alcohols, polyethylene glycols, polyhydroxyethoxylated castor oil, peanut oil, olive oil, gelatin, lactose, terra alba, sucrose, dextrin, magnesium carbonate, sugar, cyclodextrin, amylose, magnesium stearate, talc, gelatin, agar, pectin, acacia, stearic acid or lower alkyl ethers of cellulose, silicic acid, fatty acids, fatty acid amines, fatty acid monoglycerides and diglycerides, pentaerythritol fatty acid esters, polyoxyethylene, hydroxymethylcellulose and polyvinylpyrrolidone. Similarly, the carrier or diluent can include any sustained release material known in the art, such as glyceryl monostearate or glyceryl distearate, alone or mixed with a wax.

The formulations can be mixed with auxiliary agents which do not deleteriously react with the active compounds. Such additives can include wetting agents, emulsifying and suspending agents, salt for influencing osmotic pressure, buffers and/or coloring substances preserving agents, sweetening agents or flavoring agents. The compositions can also be sterilized if desired.

The route of administration can be any route which effectively transports the active compound of the invention to the appropriate or desired site of action, such as oral, nasal, pulmonary, buccal, subdermal, intradermal, transdermal or parenteral, e.g., rectal, depot, subcutaneous, intravenous, intraurethral, intramuscular, intranasal, ophthalmic solution or an ointment, the oral route being preferred.

Thus, the present compounds may be systemically administered, e.g., orally, in combination with a pharmaceutically acceptable vehicle such as an inert diluent or an assimilable edible carrier. They may be enclosed in hard or soft shell gelatin capsules, may be compressed into tablets, or may be incorporated directly with the food of the patient's diet. For oral therapeutic administration, the active compound may be combined with one or more excipients and used in the form of ingestible tablets, buccal tablets, troches, capsules, elixirs, suspensions, syrups, wafers, and the like. Such compositions and preparations should contain at least 0.1% of active compound. The percentage of the compositions and preparations may, of course, be varied and may conveniently be between about 2 to about 60% of the weight of a given unit dosage form. The amount of active compound in such therapeutically useful compositions is such that an effective dosage level will be obtained.

The tablets, troches, pills, capsules, and the like may also contain the following: binders such as gum tragacanth, acacia, corn starch or gelatin; excipients such as dicalcium phosphate; a disintegrating agent such as corn starch, potato starch, alginic acid and the like; a lubricant such as magnesium stearate; and a sweetening agent such as sucrose, fructose, lactose or aspartame or a flavoring agent such as peppermint, oil of wintergreen, or cherry flavoring may be added. When the unit dosage form is a capsule, it may contain, in addition to materials of the above type, a liquid carrier, such as a vegetable oil or a polyethylene glycol. Various other materials may be present as coatings or to otherwise modify the physical form of the solid unit dosage form. For instance, tablets, pills, or capsules may be coated with gelatin, wax, shellac or sugar and the like. A syrup or elixir may contain the active compound, sucrose or fructose as a sweetening agent, methyl and propylparabens as preservatives, a dye and flavoring such as cherry or orange flavor. Of course, any material used in preparing any unit dosage form should be pharmaceutically acceptable and substantially non-toxic in the amounts employed. In addition, the active compound may be incorporated into sustained-release preparations and devices.

Injectable dosage forms generally include aqueous suspensions or oil suspensions which can be prepared using a suitable dispersant or wetting agent and a suspending agent Injectable forms can be in solution phase or in the form of a suspension, which is prepared with a solvent or diluent. Acceptable solvents or vehicles include sterilized water, Ringer's solution, or an isotonic aqueous saline solution. Alternatively, sterile oils can be employed as solvents or suspending agents. Preferably, the oil or fatty acid is non-volatile, including natural or synthetic oils, fatty acids, mono-, di- or tri-glycerides.

The active compound may also be administered intravenously or intraperitoneally by infusion or injection. Solutions of the active compound or its salts can be prepared in water, optionally mixed with a nontoxic surfactant. Dispersions can also be prepared in glycerol, liquid polyethylene glycols, triacetin, and mixtures thereof and in oils. Under ordinary conditions of storage and use, these preparations contain a preservative to prevent the growth of microorganisms.

The pharmaceutical dosage forms suitable for injection or infusion can include sterile aqueous solutions or dispersions or sterile powders comprising the active ingredient which are adapted for the extemporaneous preparation of sterile injectable or infusible solutions or dispersions, optionally encapsulated in liposomes. In all cases, the ultimate dosage form must be sterile, fluid and stable under the conditions of manufacture and storage. The liquid carrier or vehicle can be a solvent or liquid dispersion medium comprising, for example, water, ethanol, a polyol (for example, glycerol, propylene glycol, liquid polyethylene glycols, and the like), vegetable oils, nontoxic glyceryl esters, and suitable mixtures thereof. The proper fluidity can be maintained, for example, by the formation of liposomes, by the maintenance of the required particle size in the case of dispersions or by the use of surfactants. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars, buffers or sodium chloride. Prolonged absorption of the injectable compositions can be brought about by the use in the compositions of agents delaying absorption, for example, aluminum monostearate and gelatin.

For injection, the formulation can also be a powder suitable for reconstitution with an appropriate solution as described above. Examples of these include, but are not limited to, freeze dried, rotary dried or spray dried powders, amorphous powders, granules, precipitates, or particulates. For injection, the formulations can optionally contain stabilizers, pH modifiers, surfactants, bioavailability modifiers and combinations of these. The compounds can be formulated for parenteral administration by injection such as by bolus injection or continuous infusion. A unit dosage form for injection can be in ampoules or in multi-dose containers.

Sterile injectable solutions are prepared by incorporating the active compound in the required amount in the appropriate solvent with various of the other ingredients enumerated above, as required, followed by filter sterilization. In the case of sterile powders for the preparation of sterile injectable solutions, the methods of preparation can be vacuum drying and the freeze drying techniques, which yield a powder of the active ingredient plus any additional desired ingredient present in the previously sterile-filtered solutions.

For topical administration, the present compounds may be applied in pure form, i.e., when they are liquids. However, it will generally be desirable to administer them to the skin as compositions or formulations, in combination with a dermatologically acceptable carrier, which may be a solid or a liquid.

Useful solid carriers include finely divided solids such as talc, clay, microcrystalline cellulose, silica, alumina and the like. Useful liquid carriers include water, alcohols or glycols or water-alcohol/glycol blends, in which the present compounds can be dissolved or dispersed at effective levels, optionally with the aid of non-toxic surfactants. Adjuvants such as fragrances and additional antimicrobial agents can be added to optimize the properties for a given use. The resultant liquid compositions can be applied from absorbent pads, used to impregnate bandages and other dressings, or sprayed onto the affected area using pump-type or aerosol sprayers.

Thickeners such as synthetic polymers, fatty acids, fatty acid salts and esters, fatty alcohols, modified celluloses or modified mineral materials can also be employed with liquid carriers to form spreadable pastes, gels, ointments, soaps, and the like, for application directly to the skin of the user.

The formulations of the invention can be designed to provide quick, sustained, or delayed release of the active ingredient after administration to the patient by employing procedures well known in the art. Thus, the formulations can also be formulated for controlled release or for slow release.

Compositions contemplated by the present invention can include, for example, micelles or liposomes, or some other encapsulated form, or can be administered in an extended release form to provide a prolonged storage and/or delivery effect. Therefore, the formulations can be compressed into pellets or cylinders and implanted intramuscularly or subcutaneously as depot injections. Such implants can employ known inert materials such as silicones and biodegradable polymers, e.g., polylactide-polyglycolide. Examples of other biodegradable polymers include poly(orthoesters) and poly(anhydrides).

For nasal administration, the preparation can contain a compound of the invention, dissolved or suspended in a liquid carrier, preferably an aqueous carrier, for aerosol application. The carrier can contain additives such as solubilizing agents, e.g., propylene glycol, surfactants, absorption enhancers such as lecithin (phosphatidylcholine) or cyclodextrin, or preservatives such as parabens.

For parenteral application, particularly suitable are injectable solutions or suspensions, such as aqueous solutions with the active compound dissolved.

A typical capsule for oral administration contains compounds of the invention (250 mg), lactose (75 mg) and magnesium stearate (15 mg). The mixture is passed through a 60 mesh sieve and packed into a No. 1 gelatin capsule. A typical injectable preparation is produced by aseptically placing 250 mg of compounds of the invention into a vial, aseptically freeze-drying and sealing. For use, the contents of the vial are mixed with 2 mL of sterile physiological saline, to produce an injectable preparation.

In various embodiments, the invention provides the use of a compound of the invention or of a pharmaceutical composition of the invention for treatment of a disease, disorder or injury.

The compounds of the invention are effective over a wide dosage range. Useful dosages of the compounds of formula I, II or III (or any provided herein) can be determined by comparing their in vitro activity, and in vivo activity in animal models. Methods for the extrapolation of effective dosages in mice, and other animals, to humans are known to the art; for example, see U.S. Pat. No. 4,938,949.

The amount of the compound, or an active salt or derivative thereof, required for use in treatment will vary not only with the particular salt selected but also with the route of administration, the nature of the condition being treated and the age and condition of the patient and will be ultimately at the discretion of the attendant physician or clinician.

In general, however, a suitable dose will be in the range of from about 0.5 to about 100 mg/kg, e.g., from about 10 to about 75 mg/kg of body weight per day, such as 3 to about 50 mg per kilogram body weight of the recipient per day, preferably in the range of 6 to 90 mg/kg/day, most preferably in the range of 15 to 60 mg/kg/day.

The compound is conveniently administered in unit dosage form; for example, containing 5 to 1000 mg, conveniently 10 to 750 mg, most conveniently, 50 to 500 mg of active ingredient per unit dosage form.

Ideally, the active ingredient should be administered to achieve peak plasma concentrations of the active compound of from about 0.5 to about 75 μM, preferably, about 1 to 50 μM, most preferably, about 2 to about 30 μM. This may be achieved, for example, by the intravenous injection of a 0.05 to 5% solution of the active ingredient, optionally in saline, or orally administered as a bolus containing about 1-100 mg of the active ingredient. Desirable blood levels may be maintained by continuous infusion to provide about 0.01-5.0 mg/kg/hr or by intermittent infusions containing about 0.4-15 mg/kg of the active ingredient(s).

The desired dose may conveniently be presented in a single dose or as divided doses administered at appropriate intervals, for example, as two, three, four or more sub-doses per day. The sub-dose itself may be further divided, e.g., into a number of discrete loosely spaced administrations

Generally, the compounds of the invention are dispensed in unit dosage form including from about 0.05 mg to about 1000 mg of active ingredient together with a pharmaceutically acceptable carrier per unit dosage.

Usually, dosage forms suitable for oral, nasal, pulmonal or transdermal administration include from about 125 μg to about 1250 mg, from about 250 μg to about 500 mg, and from about 2.5 mg to about 250 mg, of the compounds admixed with a pharmaceutically acceptable carrier or diluent.

Dosage forms can be administered daily, or more than once a day, such as twice or thrice daily. Alternatively dosage forms can be administered less frequently than daily.

The following examples are intended to further illustrate certain embodiments of the invention and are not intended to limit the scope of the invention in any way.

### EXAMPLES

### Example 1

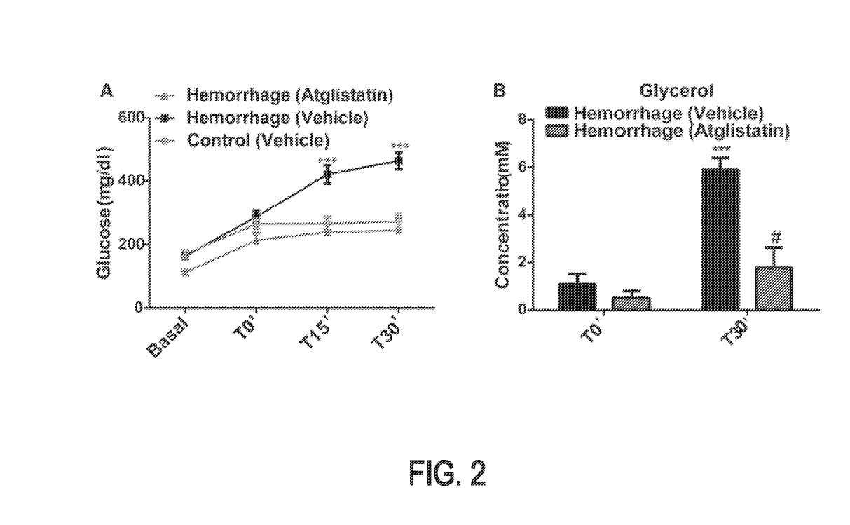

Understanding the biological basis of postoperative hyperglycemia provides a molecular target for therapeutic intervention. Surgery and anesthesia induce a neuroendocrine stress response leading to the release of counterregulatory hormones and the induction of whole body catabolism, typified by hyperglycemia. Although stress hormones decrease peripheral insulin sensitivity, it is largely hepatic glucose production that is responsible for elevations in circulating glucose (1). The mechanism underlying the increased hepatic glucose production has not been clearly understood; however, as described herein, recent studies have identified adipose tissue lipolysis and release of the gluconeogenic substrate glycerol as a required event in induction of the immediate post-operative hyperglycemia that occurs in animal models (FIG. 2). By inhibiting adipose tissue lipolysis, post-operative glucose excursions can be prevented.

In adipocytes, stress hormones, such as catecholamines and glucagon, signal through G-protein coupled receptors to activate the production of cAMP. cAMP-dependent protein kinase (PKA) signals to lipases and lipid droplet binding proteins to hydrolyze triacylglycerol to its constituent components, fatty acids and glycerol. Adipose Triacylglycerol Lipase (ATGL) is the initial lipase in the hydrolysis of triacylglycerol breakdown. Genetic loss or pharmacological inhibition of ATGL with the ATGL-specific antagonist Atglistatin largely blocks stress-induced lipolysis.

The primary mechanism for feedback inhibition of adipose lipolysis is insulin-stimulated breakdown of cAMP. The inability of T1DM patients to secrete insulin to dampen the lipolytic response contributes to episodes of diabetic ketoacidosis and this also underlies the postoperative hyperglycemia seen in diabetic patients. Similar to the findings in control mice, it is expected that in T1DM adipose tissue lipolysis will drive postoperative hyperglycemia, with the added risk of diabetic ketoacidosis (DKA).

Inhibiting Adipose Tissue Lipolysis Will Attenuate the Acute Hyperglycemia that Occurs During and Immediately after Surgery in a Mouse Model of T1DM.

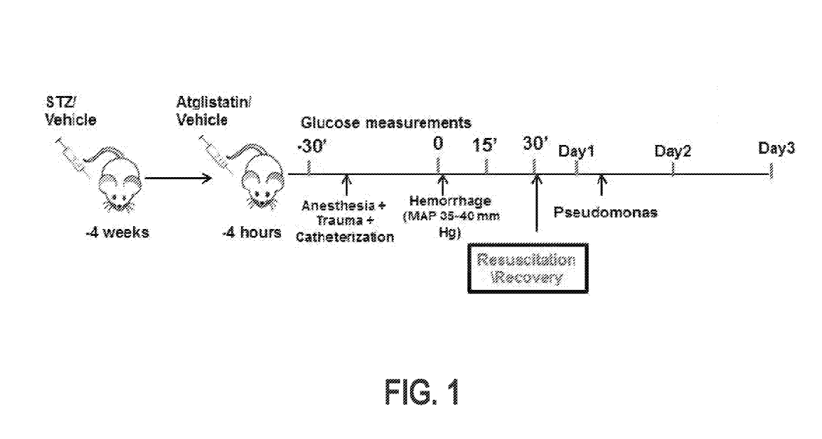

The acute changes in blood glucose that occur in a T1DM model were invesitaged. Streptozotocin (STZ)-treated mice were subjected to a hemorrhage protocol to mimic surgical induction of the neuroendocrine stress response in humans (FIG. 1). Adipose tissue lipolysis was controlled via the addition of the small molecule inhibitor Atglistatin. The extent of glycemic excursions and ketogenesis was followed.

Mouse Model of T1DM:

Separate rodent models of diabetes were employed to investigate the unique co-morbidities associated with T1DM. For the T1DM model, C57BL/6J mice were subjected to low dose injection of streptozotocin (50 mg/kg) at 6-8 weeks of age (see the website for the Diabetic Complications Consortium). This model of T1DM is considered to more closely mimic the human condition as it is only a partial insulin deficiency and while it allows hyperglycemia it does not induce ketoacidosis under basal conditions.

Surgery Model (Hemorrhage Protocol):

The protocol for modeling surgery-induced stress has been adapted from several sources and is available to an art worker. Although few human patients undergo extensive hemorrhage during elective surgeries, this rodent protocol is a consistent and reproducible experimental model for surgical stress in humans. Briefly, on the day of the surgery 10-12 week old male mice were fasted for six hours prior to surgery and pre-treated with or without lmg/kg body weight of Atglistatin at four hours prior to surgery to prevent adipose tissue lipolysis. Mice were then anesthetized by continuous inhalation of isoflurane and subjected to a 2-cm ventral midline laparotomy. The abdomen will then be sutured, a catheter was placed in the right femoral artery and mice were hemorrhaged from the left femoral artery to a mean arterial pressure (MAP) of 35-40 mm Hg (T0′). Blood glucose and ketone levels were monitored during the surgery. The mice were followed until 30′ post-hemorrhage and were maintained continuously under anesthesia. At the termination of the experiment, blood was collected to measure serum catecholamines, insulin, cytokines, ketones and glycerol levels, among other possible metabolic parameters.

The effect of Atglistatin pre-treatment on hyperglycemia in control mice is shown in FIG. 2A. FIG. 2B demonstrates the effectiveness of the Atglistatin in attenuating adipose tissue lipolysis as assessed by serum glycerol levels.

Attenuating Postoperative Glycemic Excursions Via Inhibition of Adipose Tissue Lipolysis Will Decrease Morbidities and Mortality Rates in a Mouse Model of T1DM.

How preventing hyperglycemia can aid recovery and improve survival will be investigated. STZ mice will be subjected to hemorrhage protocol described above, but then resuscitated by fluid replacement to perform a survival surgery. The metabolic status will be followed for up to three days and indices of wound repair and susceptibility to postoperative pneumonia was monitored (FIG. 1).

Control and T1DM mice, treated with Atglistatin to inhibit lipolysis or vehicle, will be subjected to the hemorrhage and resuscitation protocol. The ability of Atglistatin to improve survival under baseline conditions, as well as improve wound healing and recovery, will be determined. In addition, it will be determined whether attenuating postoperative hyperglycemia via Atglistatin administration improves survival from postoperative pseudomonal pneumonia and/or improve indices of pneumonia.

Surgery Model (Hemorrhage and Resuscitation Protocol):

Mice will be subjected to the surgery model outlined above, but at 30 min post-hemorrhage will be resuscitated using a Lactated Ringers (LR) solution at a fixed volume of 2× the volume of blood removed over a fifteen min period. After vessel ligation the incision is closed, the anesthesia will be discontinued, and the animal kept on a heated pad. To manage pain the animals will be injected locally with a subcutaneous dose of Buprenorphine. Animals will be monitored for several hours after surgery taking hourly blood glucose measurements. At 24 hours the mice will be subjected to a glucose tolerance test (GTT) to measure glucose homeostasis or subjected to further tests (below).

Wound Healing Protocol:

A subset of mice will be subjected to two full thickness excision wounds (1 cm) on the shaved and depilated dorsum on either side of the spine. At the completion of the experiment the wounds will be excised and examined histologically for immune cell infiltration and epidermal regeneration and repair (6).

Pseudomonas aeruginosa Model of Respiratory Infection:

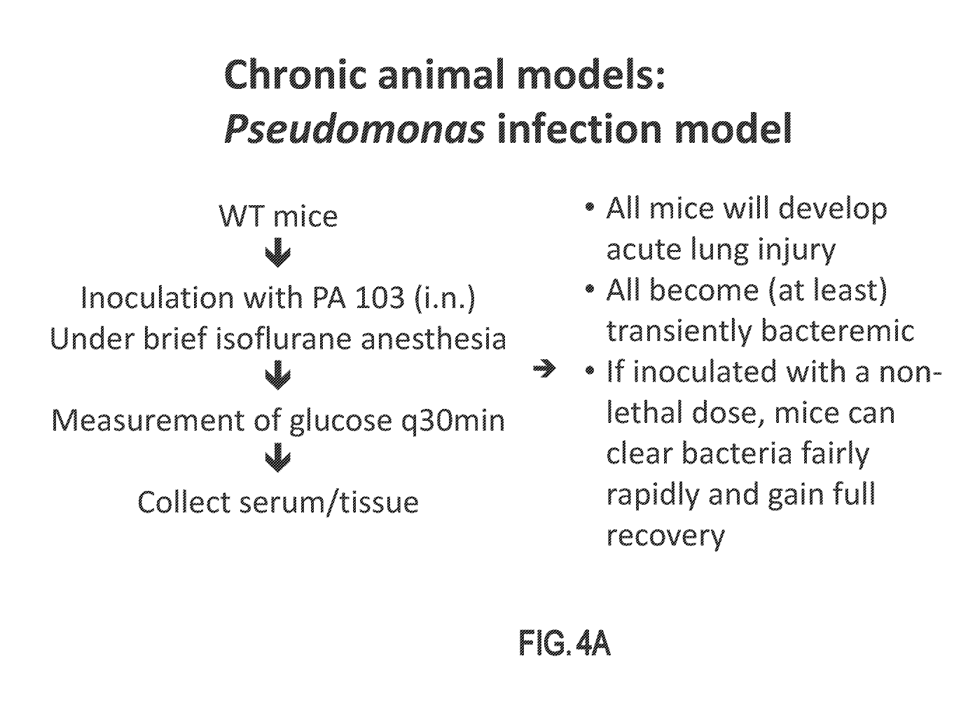

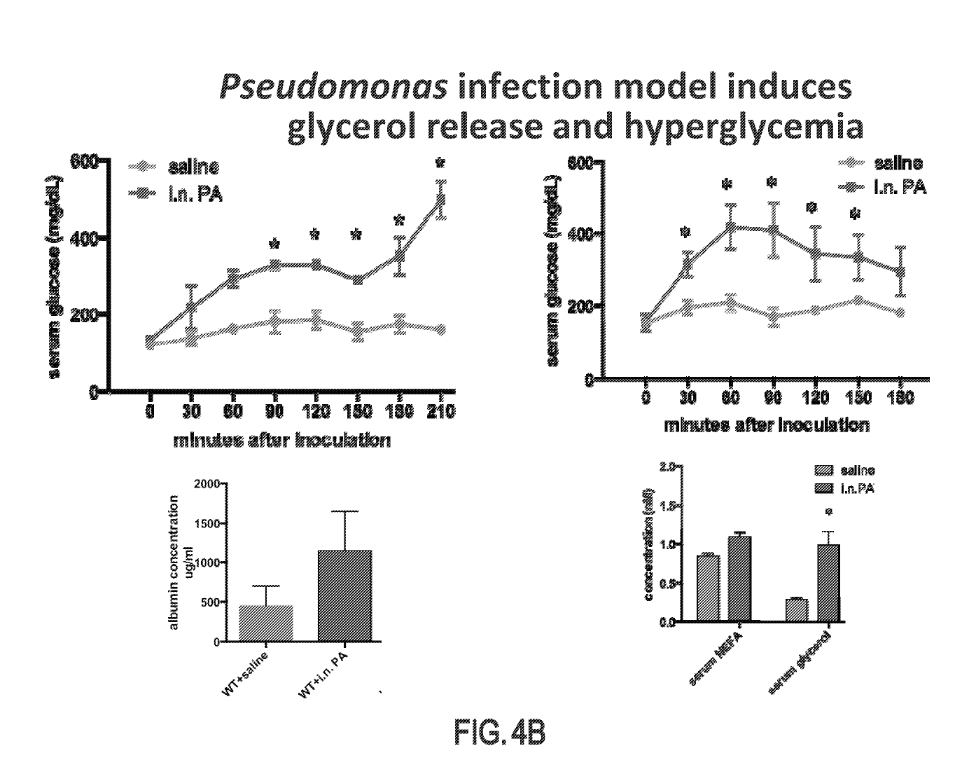

Another subset of mice will be subjected to inoculation with Pseudomonas aeroginosa PA103 serotype 011 t 24 hours after resuscitation from the hemorrhage protocol. 0.2-1.0×10⁵ CFU in 50 ul saline will be instilled intranasally. Animals will be euthanized at various time points after inoculation. Lung injury will be assessed by albumin ELISA in the bronchoalveolar lavage (BAL) fluid. Bacterial counts will be performed in the lung and blood, flow cytometric analysis of BAL cells will be done to assess for leukocyte infiltration and function, and BAL fluid as well as serum will be analyzed for inflammatory cytokine release. (FIGS. 4A-B.)

Inhibition of adipose tissue lipolysis in diabetic mice will result in improved wound healing, and decreased lung injury. Immune cell function in STZ mice will be restored to levels comparable with normoglycemic mice in response to Atglistatin treatment.

### Example 2

Rodent hemorrhage and trauma model—To activate the adaptive stress response and emulate the acute hyperglycemia and insulin resistance that develops after surgery, trauma and hemorrhage mice as previously described will be used. Briefly, 10-12 week old male mice are fasted for 4-6 hours prior to surgery and 4 hours before surgery, Atglistatin (1 mg/kg) or vehicle will be injected intraperitoneally. Mice are anesthetized by continuous inhalation of 1.5% isoflurane and 98.5% 02 throughout all procedures. After the mice are shaved, surgery will begin with a 2-cm ventral midline laparotomy. The abdomen is then sutured and the wounds bathed with 1% lidocaine to reduce pain. A catheter is placed in the right femoral artery for monitoring mean arterial pressure (MAP). To replicate the blood loss during a surgery, mice are hemorrhaged from the left femoral artery to an MAP of 35-40 mmHg over a 10-minute period. Once the MAP reaches 35-40 mmHg, the hemorrhage begins. After 30 minutes, insulin (0.5 U) or saline is injected into the inferior vena cava and tissues (subcutaneous adipose, gonadal adipose, liver, skeletal muscle) are harvested after 4 minutes. Control mice are subjected to trauma alone. Mice are subjected to anesthesia, laparotomy, and catheterization. 30 min after catheterization, insulin (0.5 U) or saline will be injected into the inferior vena cava and tissues will be harvested after 4 minutes. Blood glucose levels are monitored during the surgery.

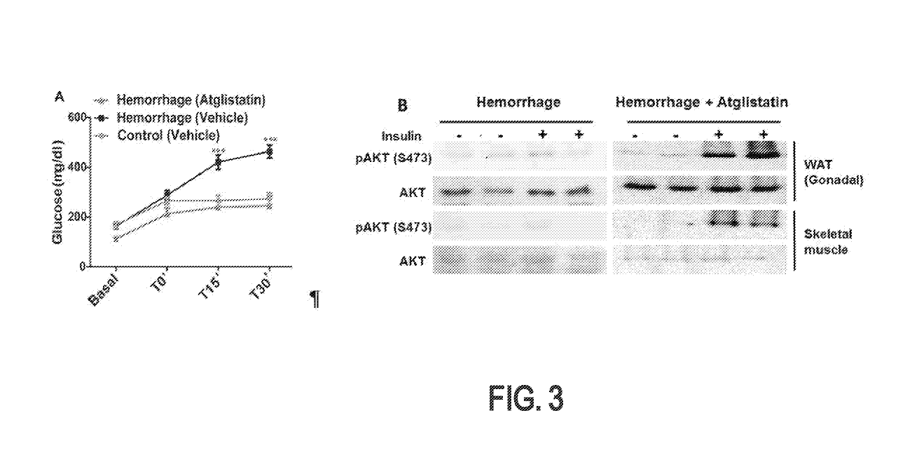

A summary of the hyperglycemia attenuation by Atglistatin is shown FIG. 3A, and the attenuation of insulin resistance in adipose tissue and skeletal muscle in FIG. 3B. Vehicle, control group are mice that were not treated with STZ and not hemorrhaged, but still subjected to anesthesia. It was observe that Atglistatin was effective in preventing hemorrhage-induced hyperglycemia. The effectiveness of the Atglistatin treatment in eliminating lipolysis is shown in the reduction in circulating glycerol.

Diabetic patients that undergo elective or emergency surgery have higher postoperative morbidity and mortality rates than nondiabetic controls. Hyperglycemia is a common occurrence following trauma or surgery, even in patients without a previous history of diabetes. It is well accepted that postoperative and post trauma hyperglycemia is associated with worse neurologic outcome, increased risk of nosocomial infections, prolonged ICU and hospital length, and increased overall mortality. Current treatment involves intensive insulin therapy; however, close and accurate monitoring of blood glucose levels is required for both healthy and especially diabetic patients due to the risk of hypoglycemia.

Provided herein is a method that would allow pharmacological inhibition of stress-induced hyperglycemia in both normal and diabetic patients independently of insulin administration. By targeting adipose tissue lipolysis immediately before surgery, glucose excursions can be prevented, thus attenuating post-operative morbidities and mortality without the risk of insulin therapy induced hypoglycemia. The results from these studies will have a significant impact on postoperative hyperglycemia and recovery in diabetic patients. More importantly, unlike insulin, the use of Atglistatin may improve the insulin resistance that occurs post-operatively.

### BIBLIOGRAPHY

1. Li L & Messina J L (2009) Acute insulin resistance following injury. Trends in endocrinology and metabolism: TEM 20(9):429-435.
2. Canadian Diabetes Association Clinical Practice Guidelines Expert C, Houlden R, Capes S, Clement M, & Miller D (2013) In-hospital management of diabetes. Canadian journal of diabetes 37 Suppl 1:S77-81.
3. Rueda A M, et al. (2010) Hyperglycemia in diabetics and non-diabetics: effect on the risk for and severity of pneumococcal pneumonia. The Journal of infection 60(2):99-105.
4. Vann M A (2009) Perioperative management of ambulatory surgical patients with diabetes mellitus. Current opinion in anaesthesiology 22(6):718-724.
5. Salim A, et al. (2006) Acute respiratory distress syndrome in the trauma intensive care unit: Morbid but not mortal. Archives of surgery 141(7):655-658.
6. Wong V W, Sorkin M, Glotzbach J P, Longaker M T, & Gurtner G C (2011) Surgical approaches to create murine models of human wound healing. Journal of Biomedicine & Biotechnology 2011:969618.
Staehr P M, et al. (2013) Reduction of free fatty acids, safety, and pharmacokinetics of oral GS-9667, an A(1) adenosine receptor partial agonist. Journal of clinical pharmacology 53(4):385-392.
Shan et al., 2013, J. Anim. Sci., 91:1247-1254, Sirtuin 1 affects the transcriptional expression of adipose triglyceride lipase in porcine adipocytes.
Mayer et al., 2015, Bioorganic & Medicinal Chemistry, 23:2904, Structure-activity studies in the development of a hydrazone based inhibitor of adipose-triglyceride lipase (ATGL).
Mayer et al., 2013, Nat. Chem. Biol. 9:12, Development of small molecule inhibitors targeting adipose triglyceride lipase.
Kumar et al. Diabetes (2010).
Green et al. FEBS Letters (1983).
Zimmerman et al., U.S. Pat. No. 8,993,509.
Dodd et al., U.S. Pat. No. 8,455,617.
Urdea et al., U.S. Pat. No. 9,034,585.
Nagy et al., Biochim Biophys Acta. 2014 Apr. 4; 1841(4):588-94. doi: 10.1016/j.bbalip.2014.01.005. Epub 2014 Jan. 16

Headings are included herein for reference and to aid in locating certain sections. These headings are not intended to limit the scope of the concepts described therein under, and these concepts may have applicability in other sections throughout the entire specification.

The terms and expressions which have been employed are used as terms of description and not of limitation, and there is no intention that in the use of such terms and expressions of excluding any equivalents of the features shown and described or portions thereof, but it is recognized that various modifications are possible within the scope of the invention claimed. Thus, it should be understood that although the present invention has been specifically disclosed by certain embodiments and optional features, modification and variation of the concepts herein disclosed may be resorted to by those skilled in the art, and that such modifications and variations are considered to be within the scope of this invention as defined by the appended claims.

The disclosures of each and every patent, patent application, and publication cited herein are hereby incorporated by reference herein to the same extent as if each individual publication was specifically and individually indicated to be incorporated by reference in its entirety.

1. A method to prevent or treat stress related adipose tissue lipolysis, insulin-resistance, and/or hyperglycemia comprising administering an effective amount of a compound of formula (III) to a subject in need thereof 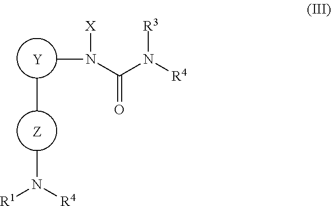
wherein R¹, R², R³, R⁴ and X are individually H, (C₁-C₆) alkyl, (C₃-C₆)cycloalkyl, (C₁-C₆)alkanoyl, trifluoromethyl, hydroxy (C₁-C₆)alkyl, aryl, aryl(C₁-C₆)alkyl;
Y and Z are selected from aryl or Het, such as heteroaryl and wherein Y and Z comprise a bond connecting two carbon atoms of Y and Z; or a pharmaceutically acceptable salt thereof.

2. A method to prevent or treat stress related adipose tissue lipolysis, insulin-resistance, and/or hyperglycemia comprising administering an effective amount of a compound of formula (II) to a subject in need thereof 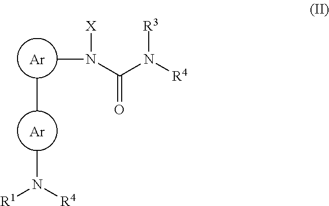
wherein R¹, R², R³, R⁴ and X are individually H, (C₁-C₆) alkyl, (C₃-C₆)cycloalkyl, (C₁-C₆)alkanoyl, trifluoromethyl, hydroxy (C₁-C₆)alkyl, aryl, aryl(C₁-C₆)alkyl; each Ar is individually selected from aryl;
or a pharmaceutically acceptable salt thereof.

3. A method to prevent or treat stress related adipose tissue lipolysis, insulin-resistance, and/or hyperglycemia comprising administering an effective amount of a compound of formula (I) to a subject in need thereof 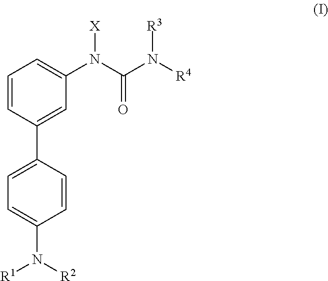
wherein R¹, R², R³, R⁴ and X are individually selected from H, (C₁-C₆) alkyl, phenyl, benzyl, phenylethyl or a pharmaceutically acceptable slat thereof.

4. A method to inhibit or treat postoperative hyperglycemia comprising administering an effective amount of a compound of formula (I), (II), or (III) to a subject in need thereof.

5. A method to improve wound healing, decrease postoperative morbidities, shorten hospital stay and/or improve mortality due to trauma and/or surgery comprising administering an effective amount of a compound of formula (I), (II), or (III) to a subject in need thereof.

6. The method of claim 1, wherein the compound is

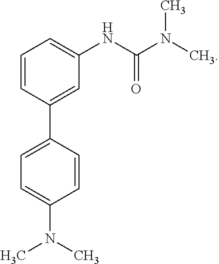 [SMILES: CN(C)C(=O)Nc1cccc(-c2ccc(N(C)C)cc2)c1]

7. The method of claim 1, wherein the subject is diabetic.

8. The method of claim 1, wherein the subject is not diabetic.

9. The method of claim 1, wherein the stress is induced by trauma.

10. The method of claim 9, wherein the trauma is a result of hemorrhage, burn, sepsis, myocardial infarction, and/or stroke.

11. The method of claim 1, wherein the stress is induced by surgery.

12. The method of claim 1, wherein the stress is induced by anesthesia.

13. The method of claim 1, wherein the compound is administered before, during and/or after the stress of claim 1 1-3 or 6-12, or the trauma and/or surgery of claim 4 or 5.

14. The method of claim 1, wherein insulin is not administered.

15. The method of claim 1, further comprising the administering of insulin to the subject.

16. A method to prevent or treat stress related adipose tissue lipolysis, insulin-resistance, and/or hyperglycemia comprising administering an effective amount of a compound of formula: 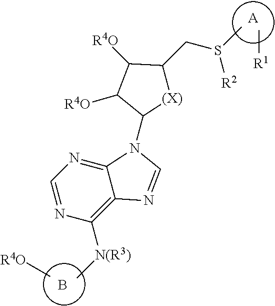
wherein A is aryl, R¹ is adjacent to (S—) and is halo, R² is absent or is (O), X is —(CH₂)—, O or S, R³ is H, phenyl, (C₁-C₆)alkyl or phenethyl, R⁴ is each individually H, (C₁-C₆)alkyl, (C₁-C₆)alkenyl, (C₁-C₆)alkynyl, B is cycloalkyl, wherein —OR⁴ is adjacent to —N, or a pharmaceutically acceptable salt of said compound.

17. The method of claim 16, wherein the compound is of the formula: 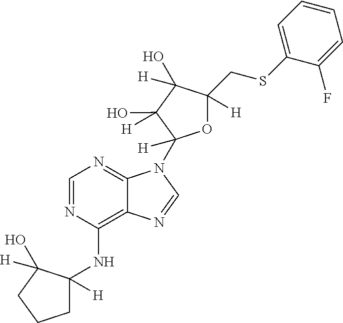 [SMILES: OC1CCCC1Nc1ncnc2c1ncn2C1OC(CSc2ccccc2F)C(O)C1O]
or a pharmaceutically acceptable salt thereof.

18. The method of claim 17, wherein the compound is 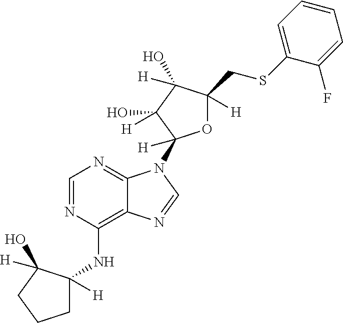 [SMILES: O[C@@H]1[C@H](O)[C@@H](CSc2ccccc2F)O[C@H]1n1cnc2c(N[C@@H]3CCC[C@H]3O)ncnc21]
or a pharmaceutically acceptable salt thereof.
